(19) United States
(12) Patent Application Publication
(10) Pub. No.: US 2025/0051314 A1
(43) Pub. Date: Feb. 13, 2025

(54) SMALL MOLECULE INHIBITORS OF INTERFERON GAMMA SIGNALING

(71) Applicants: INSTITUT CURIE, PARIS CEDEX 05 (FR); CENTRE NATIONAL DE LA RECHERCHE SCIENTIFIQUE, PARIS (FR); INSERM (INSTITUT NATIONAL DE LA SANTÉ ET DE LA RECHERCHE MÉDICALE), PARIS (FR); PARIS SCIENCES ET LETTRES, PARIS (FR)

(72) Inventors: RAPHAËL RODRIGUEZ, IVRY-SUR-SEINE (FR); CHRISTOPHE LAMAZE, PARIS (FR); CEDRIC BLOUIN, BAGNEUX (FR); LEISHEMBA KHUMAN THOIDINGJAM, PARIS (FR)

(21) Appl. No.: 18/716,962

(22) PCT Filed: Dec. 8, 2022
(86) PCT No.: PCT/EP2022/085010

§ 371 Date: Jun. 6, 2024

(30) Foreign Application Priority Data
Dec. 9, 2021 (EP) 21306735.8

(51) Int. Cl. C07D 405/06; A61K 31/515

(52) U.S. Cl. CPC C07D 405/06; A61K 31/515

(57) ABSTRACT

The present invention relates to SMIFH2 derivative compounds and their use as inhibitor of interferon-γ mediated signaling. In particular, the invention relates to compounds of general formula (I), and their use as inhibitor of interferon-γ mediated signaling, preferably for use for preventing and/or treating diseases associated to the hyper-activation of interferon-γ mediated JAK/STAT signaling.

### FIELD OF THE INVENTION

The present invention is in the field of medicine. The present invention relates to new SMIFH2 derivative compounds and their use as drugs.

### BACKGROUND OF THE INVENTION

Interferons (IFNs) are pleiotropic cytokines that play key roles in innate and adaptive immunity for host defense against intracellular infections and tumor control (Lamaze, C.; Blouin, C. Front. Immunol. 2013, 4, 267. https://doi.org/10.3389/fimmu.2013.00267). IFN binding to the type I and type II IFN receptors classically triggers a downstream activation of the canonical JAK/STAT signaling pathway, and its dysregulation has been involved in the pathogenesis of autoimmune and inflammatory diseases, and cancer (Benci, J. L. et al. Cell 2016, 167 (6), 1540-1554.e12. https://doi.org/10.1016/j.cell.2016.11.022.). Thus, targeting IFN signaling pathways represents an attractive therapeutic strategy to treat these indications. Currently, the most effective approach to block JAK/STAT signaling is based on the use of JAK tyrosine kinase inhibitors (Jakinibs) (Villarino, A. V. Kanno, Y.; O'Shea, J. J. Nat. Immunol. 2017, 18 (4), 374-384. https://doi.org/10.1038/ni.3691). These small molecules have shown promising results for the treatment of dysregulated immune responses in various pathologies. However, since JAK tyrosine kinase can be activated by cytokines and growth factors, current inhibitors block with no specificity the signaling downstream of these inducers, which are involved in many important physiological functions. Jakinibs therefore suffer from a lack of specificity for a given signaling pathway and therefore exhibit important side-effects (Banerjee, S. et al. Drugs 2017, 77 (5), 521-546. https://doi.org/10.1007/s40265-017-0701-9). Another strategy to target IFNγ selectively includes the use of a monoclonal antibody termed Emapalumab (Hatterer, E. et al. 10th Jt. Meet. Int. Cytokine Soc. Int. Soc. Interferon Cytokine Res. 2012, 59 (3), 570. https://doi.org/10.1016/j.cyto.2012.06.257). However, its moderate efficacy has yet prevented marketing authorization in Europe for the treatment of HLH (Dimitrova, E. K. Gamifant https://www.ema.europa.eu/en/medicines/human/EPAR/gamifant (accessed 2021 Aug. 18)). Developing selective inhibitors of IFNγ-activated JAK/STAT signaling remains a challenging endeavor. Such pharmacological approaches would enable to dissect the complex biology of IFNγ in various settings in greater detail and could lead to the development of new therapeutics for the treatment of specific cancers, autoimmune and inflammatory diseases, atherosclerosis and other metabolic syndromes.

### SUMMARY OF THE INVENTION

The inventors discovered SMIFH2 is capable of inhibiting IFNγ signaling and in particular IFNγ-induced JAK/STAT activation, a major signaling pathway involved in many diseases including inflammatory and auto-immune diseases as well as a subset of cancers. They identified several SMIFH2 derivative compounds with improved properties.

More particularly, the SMIFH2 derivative compounds of the invention present several advantages in first-line treatments of autoimmune and inflammation diseases:
Specificity: The SMIFH2 derivative compounds are highly specific and some of them exclusively block IFN-γ activated JAK/STAT signaling pathway.
Less expensive: SMIFH2 derivatives are small molecules for which production costs are lower, and production rate much higher than for antibodies. SMIFH2 derivatives of the invention are then the first affordable small molecules that specifically targets JAK/STAT signaling downstream of IFN-γ.
Reduced side effects: Small molecules are not immunogenic and therefore show less immunity-related side effects. This is particularly interesting for diseases that imply chronic treatments such as systemic lupus erythematosus (SLE) or Crohn's disease.
Pharmacomodulation: Small molecules can be easily and rapidly customized to improve their stability, target accessibility or for being delivered as prodrugs.
BBB permeant: Small molecules have the capability to pass through the blood brain barrier (BBB). This property could be beneficial since the SMIFH2 derivatives of the invention can be used to impede the potential deleterious roles of IFN-γ in central nervous system.

Accordingly, the invention relates to compound of the following general formula (I):

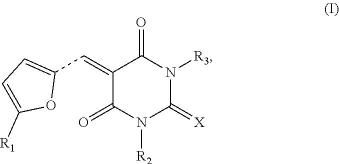

or a pharmaceutically acceptable salt, stereoisomer, tautomer or solvate thereof,
for use as inhibitor of interferon-γ mediated signaling, preferably for use for preventing and/or treating diseases associated to the hyper-activation of interferon-γ mediated JAK/STAT signaling,
wherein,
   X is an oxygen or sulfur atom;
   R₁ is a hydrogen atom, halo, nitro (NO₂), (C₁-C₆)alkyl group or (C₂-C₆)alkynyl group;
   R₂ and R₃ are, independently of one another, a hydrogen atom, a (C₀-C₆)alkyl-ethynyl (—(CH₂)₀₋₆—C≡CH); a (C₀-C₃)alkyl-NH—C(O)—R′; a (C₀-C₃)alkyl-C(O)—NR′R″; a (C₀-C₃)alkyl-NH—C(O)—OR′; a (C₀-C₃)alkyl-NH—C(O)—NR′R″; a (C₀-C₃)alkyl-C(O)—R′; a (C₀-C₃)alkyl-C(O)—OR′; a (C₀-C₃)alkyl-NR′R″; a (C₀-C₃)alkyl-SOR′; a (C₀-C₃)alkyl-SO₂R′; a (C₀-C₃)alkyl-SONR′R″; a (C₀-C₃)alkyl-SO₂NR′R″; (C₀-C₃)alkyl-NHSO₂R′; or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)heterocycloalkyl group, (C₆-C₁₂)aryl group, or (C₅-C₁₂)heteroaryl group; said group being optionally substituted by at least one R;
R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group; and
R′ and R″ are independently selected from the group consisting of an hydrogen atom or a (C₁-C₆)alkyl group optionally substituted by at least one halogen.

The invention also relates to a compound of general formula (I), or a pharmaceutically acceptable salt, stereoisomer, tautomer or solvate thereof, wherein,
X is an oxygen or sulfur atom;
R₁ is a chlorine atom or a (C₂-C₆)alkynyl group;
R₂ and R₃ are, independently of one another, a hydrogen atom, a (C₀-C₆)alkyl-ethynyl (—(CH₂)₀₋₆—C≡CH); a (C₀-C₃)alkyl-NH—C(O)—R′; a (C₀-C₃)alkyl-C(O)—NR′R″; a (C₀-C₃)alkyl-NH—C(O)—OR′; a (C₀-C₃)alkyl-NH—C(O)—NR′R″; a (C₀-C₃)alkyl-C(O)—R′; a (C₀-C₃)alkyl-C(O)—OR′; a (C₀-C₃)alkyl-NR′R″; a (C₀-C₃)alkyl-SOR′; a (C₀-C₃)alkyl-SO₂R′; a (C₀-C₃)alkyl-SONR′R″; a (C₀-C₃)alkyl-SO₂NR′R″; (C₀-C₃)alkyl-NHSO₂R′; or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)heterocycloalkyl group, (C₆-C₁₂)aryl group, or (C₅-C₁₂)heteroaryl group; said group being optionally substituted by at least one R;
R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group; and
R′ and R″ are independently selected from the group consisting of an hydrogen atom or a (C₁-C₆)alkyl group optionally substituted by at least one halogen;
provided that said compound is not a compound (Ia) or (Ib):

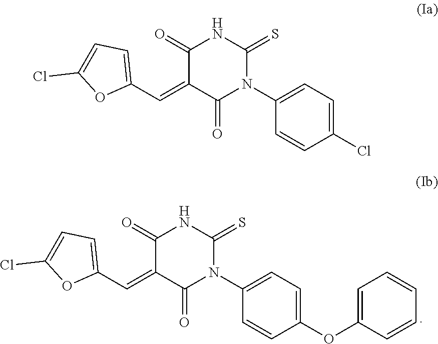 [SMILES: O=C1NC(=S)N(c2ccc(Cl)cc2)C(=O)/C1=C/c1ccc(Cl)o1 ; O=C1NC(=S)N(c2ccc(Oc3ccccc3)cc2)C(=O)/C1=C/c1ccc(Cl)o1]

The invention also relates to a compound as defined above, for use as a drug, to a pharmaceutical composition comprising such a compound and optionally a pharmaceutically acceptable carrier, to this pharmaceutical composition for use as a drug, and to the use of such a compound or pharmaceutical composition for the manufacture of a medicine.

The invention also relates to the non-therapeutic use of a compound selected from the group consisting of compounds 5l, 6a, 6d, 6i, 6j, 6k, 6l and 6m as defined herein, preferably 6a, 6d, 6i, 6j, 6k and 6l, as inhibitor of formin FH2 domains.

### BRIEF DESCRIPTION OF THE FIGURES

FIG. 1: Anti-IFNγ activity screening performed using immunoblot analysis of pSTAT1 and tSTAT1 levels in Hela cells treated with IFNγ, preincubated with small molecules (40 μM) for 20 min as indicated.

FIG. 2: Anti-actin polymerization activity screening performed using wide-field microscopy images of Hela cells treated with small molecules (40 μM) for 20 min and stained for actin with phalloidin-Alexa488. Bar=30 μm. Quantification of cell area. Mean value±SD. Statistical analysis with one-way ANOVA. n=3 independent experiments.

FIG. 3: (A) Immunoblot analysis of pSTAT1 and tSTAT1 levels in Hela cells treated by IFNγ, pre-incubated or not with different concentrations of indicated compounds for 20 min. Tubulin serves as a loading control. (B) Quantification as ratio of control of immunoblot in (A). IC50 towards IFNγ are determined by non-linear curve fit. n=3 independent experiments. (C) Dose-response viability curves of Hela cells treated for 24 h with specified compounds. IC50 are determined by non-linear curve fit. n=3 independent experiments. Therapeutic Index=(IC50 of Cell Viability/IC50 of pSTAT1 inhibition). D) Dose-response viability curves of Hela cells treated for 24 h with specified compounds. IC50 are determined by non-linear curve fit. n=3 independent experiments. Therapeutic Index=(IC50 of Cell Viability/IC50 of pSTAT1 inhibition). IC50 and therapeutic index values for compounds have been regrouped in a table (Table 5).

### DETAILED DESCRIPTION OF THE INVENTION

### Definitions

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt” is intended to mean, in the framework of the present invention, a salt of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(2) salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine. N-methylglucamine, triethanolamine, tromethamine and the like. Acceptable inorganic bases comprise aluminium hydroxide, calcium hydroxide, potassium hydroxide, sodium carbonate and sodium hydroxide.

The “stereoisomers” are isomeric compounds that have the same molecular formula and sequence of bonded atoms, but differ in the 3D-dimensional orientations of their atoms in space. The stereoisomers include enantiomers, diastereoisomers, Cis-trans and E-Z isomers, conformers, and anomers. In a preferred aspect of the disclosure, the stereoisomers include diastereoisomers and enantiomers.

The “tautomers” are isomeric compounds that differ only in the position of the protons and the electrons.

The “solvates” of the present disclosure include conventional solvates such as those formed during the last step of the preparation of the compounds of the invention due to the presence of solvents. It can be for example an hydrate or an alcoholate such as an ethanolate.

The term “halogen” or “halo”, as used in the invention, refers to a fluorine, bromine, chlorine or iodine atom.

The term “Cₓ-C_{y}” in which x and y are integers, as used in the present disclosure, means that the corresponding hydrocarbon chain comprises from x to y carbon atoms. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms. The term “C₀” thus means that no hydrocarbon chain is present but only a single bond. 30 The term “alkyl”, as used in the invention, refers to a monovalent linear or branched saturated hydrocarbon chain. For example, the term “C₁-C₃alkyl” more specifically means methyl, ethyl, n-propyl, or isopropyl. The term “C₁-C₆alkyl” more specifically means methyl, ethyl, n-propyl, isopropyl, n-butyl, iso-butyl, sec-butyl, tert-butyl, pentyl or linear or branched hexyl.

The term “alkoxy” or “alkyloxy”, as used in the invention, refers to an alkyl group as defined above bound to the molecule via an oxygen atom. C₁-C₃alkoxy includes methoxy, ethoxy, propyloxy, and isopropyloxy. C₁-C₆alkoxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is a methoxy.

The term “alkenyl”, as used in the invention, refers to a straight or branched monovalent unsaturated hydrocarbon chain comprising at least one double bond including, but not limited to, ethenyl, propenyl, butenyl, pentenyl, hexenyl and the like.

The term “alkynyl”, as used in the invention, refers to a straight or branched monovalent unsaturated hydrocarbon chain comprising at least one triple bond including, but not limited to, ethynyl, propynyl, butynyl, pentynyl, hexynyl and the like. Preferably, an alkynyl group as used in the present disclosure comprises one triple bond.

In particular, the term (C₀-C₆)alkyl-ethynyl, as used in the present disclosure, refers to an alkynyl as defined above comprising one terminal triple bond; i.e. a (C₀-C₆)alkyl terminally substituted by an ethynyl.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl, preferably cyclopropyl. The term “spirocycloalkyl” includes for instance a spirocyclopentyl. In a particular aspect, the term “cycloalkyl” corresponds to a saturated monocycloalkyl group comprising between 3 and 7 atoms of carbons. In a particular aspect, the cycloalkyl group is cyclohexyl.

The term “heterocycloalkyl” corresponds to a saturated or unsaturated cycloalkyl group as above defined further comprising at least one heteroatom such as nitrogen, oxygen, or sulphur atom. It also includes fused, bridged, or spiro-connected heterocycloalkyl groups. Representative heterocycloalkyl groups include, but are not limited to 3-dioxolane, benzo[1,3]dioxolyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydro-2H-pyranyl, tetrahydrofuranyl, and tetrahydrothiophenyl. The term “heterocycloalkyl” may also refer to a 5-10 membered bridged heterocyclyl such as 7-oxabicyclo[2,2,1]heptanyl.

The term “aryl” corresponds to a mono- or bi-cyclic aromatic hydrocarbons having from 6 to 12 carbon atoms. For instance, the term “aryl” includes phenyl, biphenyl, or naphthyl. In a preferred embodiment, the aryl is a phenyl.

The term “heteroaryl” as used herein corresponds to an aromatic, mono- or poly-cyclic group comprising between 5 and 14 atoms and comprising at least one heteroatom such as nitrogen, oxygen or sulphur atom. Examples of such mono- and poly-cyclic heteroaryl group may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, B-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzisoxazolyl, oxindolyl, benzoxazolinyl, benzothienyl, benzothiazolyl, isatinyl, dihydropyridyl, pyrimidinyl, s-triazinyl, oxazolyl, or thiofuranyl. In a preferred embodiment, the heteroaryl group is a thiophenyl, a pyridinyl, a pyrazinyl, or a thiazolyl.

The expression “substituted by at least” or “substituted by” means that the group is substituted by one or several substituents of the list. For instance, the expression “a C₁-C₆alkyl substituted by at least one halogen” or “a C₁-C₆alkyl substituted by a halogen” may include a fluoromethyl (—CH₂F), a difluoromethyl (—CHF₂), or a trifluoromethyl (—CF₃).

The expression “optionally substituted” means that the group is not substituted or is substituted by one or several substituents of the list.

By “—CO—” or “—C(O)—”, it refers to an oxo group. By “—SO—” or “—S(O)—”, it refers to a sulfinyl group. By “—SO₂—” or “—S(O₂)—”, it refers to a sulfonyl group.

As used herein, the terms “subject”, “individual” or “patient” are interchangeable and refer to an animal, preferably to a mammal, even more preferably to a human, including adult and child. However, the term “subject” can also refer to non-human animals, in particular mammals such as dogs, cats, horses, cows, pigs, sheep and non-human primates, among others.

Within the context of the present disclosure, the term treatment denotes curative, symptomatic, and preventive treatment. Pharmaceutical compositions, kits, products and combined preparations of the invention can be used in humans with a disease or disorder. The pharmaceutical compositions, kits, products and combined preparations of the invention will not necessarily cure the patient but will delay or slow the progression or prevent further progression of the disease or disorder, and/or ameliorating thereby the patients' condition. In treating the disease or disorder, the pharmaceutical composition of the invention is administered in a therapeutically effective amount.

Whenever within this whole specification “treatment of a disease or disorder” or the like is mentioned with reference to the pharmaceutical composition of the invention, there is meant: a) a method for treating a disease or disorder, said method comprising administering a therapeutically effective amount of a compound of the invention or of a pharmaceutical composition comprising said compound to a subject in need of such treatment; b) the use of a compound of the invention or of a pharmaceutical composition comprising said compound for the treatment of a disease or disorder; c) the use of a compound of the invention or of a pharmaceutical composition comprising said compound for the manufacture of a medicament for the treatment of a disease or disorder; and/or d) a compound of the invention or of a pharmaceutical composition comprising said compound for use in the treatment a disease or disorder.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

By “therapeutically effective amount”, it is meant the quantity of the pharmaceutical composition of the invention which prevents, removes or reduces the deleterious effects of a disease or disorder in mammals, including humans, alone or in combination with the other active ingredients of the pharmaceutical composition, kit, product or combined preparation. It is understood that the administered dose may be lower for each compound in the composition to the “therapeutic effective amount” define for each compound used alone or in combination with other treatments than the combination described here. The “therapeutic effective amount” of the composition will be adapted by those skilled in the art according to the patient, the pathology, the mode of administration, etc.

As used herein, the term “pharmaceutically acceptable excipient” refers to any ingredient except active ingredients which are present in a pharmaceutical composition. Its addition may be aimed to confer a particular consistency or other physical or gustative properties to the final product. A pharmaceutically acceptable excipient must be devoid of any interaction, in particular chemical, with the active ingredients.

### Compounds for Use as Inhibitor of Interferon-γ Mediated Signaling

As mentioned, the invention relates to a compound of the following general formula (I):

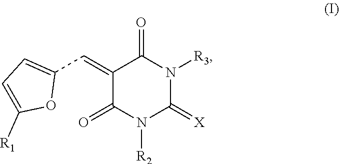

or a pharmaceutically acceptable salt, stereoisomer, tautomer or solvate thereof,
for use as inhibitor of interferon-γ mediated signaling, preferably for use for preventing and/or treating diseases associated to the hyper-activation of interferon-γ mediated JAK/STAT signaling,
wherein,
   X is an oxygen or sulfur atom;
   R₁ is a hydrogen atom, halo, nitro (NO₂), (C₁-C₆)alkyl group or (C₂-C₆)alkynyl group;
   R₂ and R₃ are, independently of one another, a hydrogen atom, a (C₀-C₆)alkyl-ethynyl (—(CH₂)₀₋₆—C≡CH); a (C₀-C₃)alkyl-NH—C(O)—R′; a (C₀-C₃)alkyl-C(O)—NR′R″; a (C₀-C₃)alkyl-NH—C(O)—OR′; a (C₀-C₃)alkyl-NH—C(O)—NR′R″; a (C₀-C₃)alkyl-C(O)—R′; a (C₀-C₃)alkyl-C(O)—OR′; a (C₀-C₃)alkyl-NR′R″; a (C₀-C₃)alkyl-SOR′; a (C₀-C₃)alkyl-SO₂R′; a (C₀-C₃)alkyl-SONR′R″; a (C₀-C₃)alkyl-SO₂NR′R″; (C₀-C₃)alkyl-NHSO₂R′; or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)heterocycloalkyl group, (C₆-C₁₂)aryl group, or (C₅-C₁₂)heteroaryl group; said group being optionally substituted by at least one R;
   R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group; and
   R′ and R″ are independently selected from the group consisting of an hydrogen atom or a (C₁-C₆)alkyl group optionally substituted by at least one halogen.

In a specific aspect, the invention relates to the compound of general formula (I) for use as defined above, in which R₁ is an halo, a (C₁-C₆)alkyl group or a (C₂-C₆)alkynyl group. In a particular aspect, R₁ is an halo selected from the group consisting of chlorine, fluorine and bromine, a (C₁-C₆)alkyl group or a (C₀-C₄)alkyl-ethynyl (—(CH₂)₀₋₄—C≡CH). In a more specific aspect, R₁ is an halo selected from the group consisting of chlorine, fluorine and bromine; a (C₁-C₃)alkyl group; or a (C₀-C₄)alkyl-ethynyl (—(CH₂)₀₋₄—C≡CH). In a more specific aspect, R₁ is an halo selected from the group consisting of chlorine, fluorine and bromine; a methyl group; or a (C₀-C₄)alkyl-ethynyl (—(CH₂)₀₋₄—C≡CH). Preferably, R₁ is a chlorine, a methyl, or a (C₀-C₄)alkyl-ethynyl (—(CH₂)₀₋₄—C≡CH), preferably an ethynyl. In a very specific aspect of the invention, R₁ is a chlorine or an ethynyl.

In a specific aspect, the invention relates to the compound of general formula (I) for use as defined above, in which X is a sulfur atom.

In a specific aspect, the invention relates to the compound of general formula (I) for use as defined above, in which X is a sulfur atom and R₁ is an halo selected from the group consisting of chlorine, fluorine and bromine; a (C₁-C₃)alkyl group; or a (C₀-C₄)alkyl-ethynyl (—(CH₂)₀₋₄—C≡CH). In a more specific aspect, X is a sulfur atom and R₁ is an halo selected from the group consisting of chlorine, fluorine and bromine; a methyl group; or a (C₀-C₄)alkyl-ethynyl (—(CH₂)₀₋₄—C≡CH). Preferably, X is a sulfur atom and R₁ is a chlorine, a methyl, or a (C₀-C₄)alkyl-ethynyl (—(CH₂)₀₋₄—C≡CH), preferably an ethynyl. In a very specific aspect of the invention, X is a sulfur atom and R₁ is a chlorine or an ethynyl.

In a specific aspect, the invention relates to the compound of general formula (I) for use as defined above, in which R₂ and R₃ are, independently of one another, a hydrogen atom or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)cycloheteroalkyl group, (C₆-C₁₂)aryl group, and (C₅-C₁₂)heteroaryl group; said group being optionally substituted by at least one R; provided that R₃ is not a hydrogen atom. In a more specific aspect, R₂ and R₃ are, independently of one another, a hydrogen atom or a group selected from (C₆-C₁₂)aryl group and (C₅-C₁₂)heteroaryl group; said group being optionally substituted by at least one R; provided that R₃ is not a hydrogen atom. In a more specific aspect, R₂ is H or a (C₆-C₁₂)aryl group optionally substituted by at least one R; and R₃ is a (C₆-C₁₂)aryl group optionally substituted by at least one R. In another more specific aspect, R₂ is H or a phenyl optionally substituted by at least one R; and R₃ is a phenyl optionally substituted by at least one R. Preferably, R₂ is H or a (C₆-C₁₂)aryl group substituted by at least one R; and R₃ is a (C₆-C₁₂)aryl group substituted by at least one R. More preferably, R₂ is H or a phenyl substituted by at least one R; and R₃ is a phenyl substituted by at least one R. More preferably, R₂ and R₃ are, independently of one another, a (C₆-C₁₂)aryl group substituted by at least one R, in particular by one or two R. Still more preferably, R₂ and R₃ are, independently of one another, a phenyl substituted by at least one R, in particular by one or two R. In a particular aspect, R₃ is preferably a phenyl group substituted by at least one R, in particular by one or two R. More particularly, R₃ is preferably a phenyl group substituted by at least one R, in particular by one or two R, and R₂ is and hydrogen atom or is identical to R₃.

In a specific aspect, the invention relates to the compound of general formula (I) for use as defined above, in which R₂ and R₃ are identical and are as defined above.

In a specific aspect, the invention relates to the compound of general formula (I) for use as defined above, in which:
X is a sulfur atom;
R₁ is an halo selected from the group consisting of chlorine, fluorine and bromine; a (C₁-C₃)alkyl group; or a (C₀-C₄)alkyl-ethynyl (—(CH₂)₀₋₄—C≡CH); and
R₂ and R₃ are, independently of one another, a hydrogen atom or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)cycloheteroalkyl group, (C₆-C₁₂)aryl group, and (C₅-C₁₂)heteroaryl group; said group being optionally substituted by at least one R;
provided that R₃ is not a hydrogen atom.

In a more specific aspect, the invention relates to the compound of general formula (I) for use as defined above, in which:
X is a sulfur atom;
R₁ is an halo selected from the group consisting of chlorine, fluorine and bromine; a methyl group; or a (C₀-C₄)alkyl-ethynyl (—(CH₂)₀₋₄—C≡CH); and
R₂ and R₃ are, independently of one another, a hydrogen atom or a group selected from (C₆-C₁₂)aryl group and (C₅-C₁₂)heteroaryl group; said group being optionally substituted by at least one R;
provided that R₃ is not a hydrogen atom.

In a more specific aspect, the invention relates to the compound of general formula (I) for use as defined above, in which:
X is a sulfur atom;
R₁ is a chlorine, a methyl, or a (C₀-C₄)alkyl-ethynyl (—(CH₂)₀₋₄—C≡CH), preferably an ethynyl;
R₂ is H or a (C₆-C₁₂)aryl group optionally substituted by at least one R; and
R₃ is a (C₆-C₁₂)aryl group optionally substituted by at least one R.

In a more specific aspect, the invention relates to the compound of general formula (I) for use as defined above, in which:
X is a sulfur atom;
R₁ is a chlorine or an ethynyl;
R₂ is H or a (C₆-C₁₂)aryl group, preferably a phenyl, substituted by at least one R; and
R₃ is a (C₆-C₁₂)aryl group, preferably a phenyl, substituted by at least one R.

In this aspect, R₂ and R₃ are preferably identical and are both a (C₆-C₁₂)aryl group, preferably a phenyl, substituted by at least one R. More preferably, R₂ and R₃ are identical and are both a phenyl group substituted by one or two R.

In a still more specific aspect, the invention relates to the compound of general formula (I) for use as defined above, in which:
X is a sulfur atom;
R₁ is an ethynyl;
R₂ is H or a (C₆-C₁₂)aryl group, preferably a phenyl, substituted by at least one R; and
R₃ is a (C₆-C₁₂)aryl group, preferably a phenyl, substituted by at least one R.

In another still more specific aspect, the invention relates to the compound of general formula (I) for use as defined above, in which:
X is a sulfur atom;
R₁ is a chlorine;
R₂ is H or a (C₆-C₁₂)aryl group, preferably a phenyl, substituted by at least one R; and
R₃ is a (C₆-C₁₂)aryl group, preferably a phenyl, substituted by at least one R selected from the group consisting of fluorine and —CF₃, preferably fluorine.

In the definitions of R₂ and R₃, R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group. In a specific aspect, R is independently selected from the group consisting of a halo selected in the group consisting of fluorine, bromine and chlorine; —CF₃; or a (C₁-C₆)alkyloxy group. In a more specific aspect, R is independently selected from the group consisting of fluorine, bromine, —CF₃, or a (C₁-C₃)alkyloxy group. Preferably, R is independently selected from the group consisting of fluorine, bromine, —CF₃, or a methoxy group (—OCH₃).

In the definitions, when R₂ and/or R₃ are a group substituted by more than one R, the R may be the same or different, preferably the R are identical.

In a very specific aspect, R₃ is a phenyl substituted by:
one substituent selected from the group consisting of bromine, fluorine, and —CF₃, in position ortho, meta or para, preferably ortho or meta;
two substituents selected from the group consisting of bromine, fluorine, and —CF₃, especially two fluorines or two-CF₃ groups, for instance either both in position meta or one in position ortho and the other in position meta.

In a very specific aspect, R₂ is H or a phenyl optionally substituted by:
one substituent selected from the group consisting of bromine, fluorine, and —CF₃, in position ortho, meta or para, preferably ortho or meta;
two substituents selected from the group consisting of bromine, fluorine, and —CF₃, especially two fluorines or two-CF₃ groups, for instance either both in position meta or one in position ortho and the other in position meta.

In an advantageous aspect of the invention, the compound for use as defined herein is selected from the group consisting of compounds 5a, 5b, 5c, 5d, 5f, 5g, 5h, 5i, 5j, 5k, 5e, 5l, 6b, 6m and SMIFH2, as defined in the following Table 1. Preferably, the compound for use as defined above has a specific activity as inhibitor of interferon-γ mediated signaling, especially it is devoid of activity of inhibiting formin FH2 domains, and/or actin polymerization and/or formin-mediated actin nucleation and/or formin-mediated elongation of actin filaments. In this preferred aspect, the compound is selected from the group consisting 5 of compounds 5a, 5b, 5c, 5d, 5f, 5g, 5h, 5i, 5j, 5k, 5l, and 6b.

TABLE 1

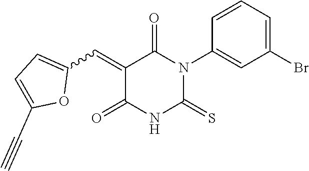 [SMILES: C#Cc1ccc(C=C2C(=O)NC(=S)N(c3cccc(Br)c3)C2=O)o1]

Compound 5a
(1-(3-bromophenyl)-5-((5-ethynylfuran-2-
yl)methylene)-2-thioxodihydropyrimidine-
4,6(1H,5H)-dione)

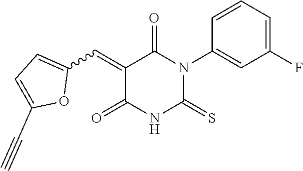 [SMILES: C#Cc1ccc(C=C2C(=O)NC(=S)N(c3cccc(F)c3)C2=O)o1]

Compound 5b
5-((5-ethynylfuran-2-
yl)methylene)-1-(3-
fluorophenyl)-2-
thioxodihydropyrimidine-
4,6(1H,5H)-dione

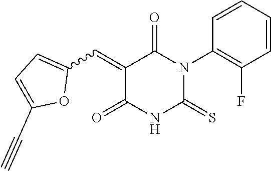 [SMILES: C#Cc1ccc(C=C2C(=O)NC(=S)N(c3ccccc3F)C2=O)o1]

Compound 5c
5-((5-ethynylfuran-2-
yl)methylene)-1-(2-
fluorophenyl)-2-
thioxodihydropyrimidine-
4,6(1H,5H)-dione

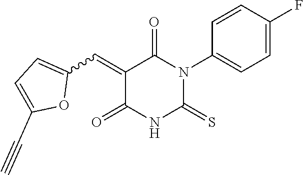 [SMILES: C#Cc1ccc(C=C2C(=O)NC(=S)N(c3ccc(F)cc3)C2=O)o1]

Compound 5d
5-((5-ethynylfuran-2-yl)methylene)-1-(4-
fluorophenyl)-2-thioxodihydropyrimidine-
4,6(1H,5H)-dione

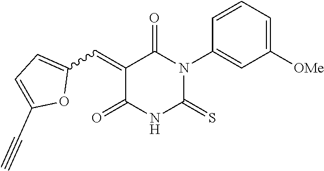 [SMILES: C#Cc1ccc(C=C2C(=O)NC(=S)N(c3cccc(OC)c3)C2=O)o1]

Compound 5f
5-((5-ethynylfuran-2-
yl)methylene)-1-(3-
methoxyphenyl)-2-
thioxodihydropyrimidine-
4,6(1H,5H)-dione

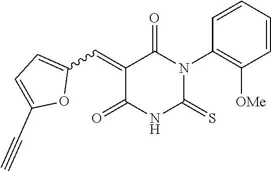 [SMILES: C#Cc1ccc(C=C2C(=O)NC(=S)N(c3ccccc3OC)C2=O)o1]

Compound 5g
5-((5-ethynylfuran-2-
yl)methylene)-1-(2-
methoxyphenyl)-2-
thioxodihydropyrimidine-
4,6(1H,5H)-dione

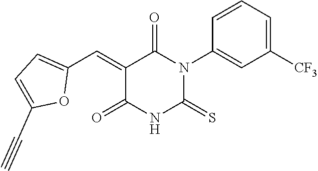 [SMILES: C#Cc1ccc(/C=C2\C(=O)NC(=S)N(c3cccc(C(F)(F)F)c3)C2=O)o1]

Compound 5h
5-((5-ethynylfuran-2-yl)methylene)-2-
thioxo-1-(3-
(trifluoromethyl)phenyl)dihydropyrimidine-
4,6(1H,5H)-dione

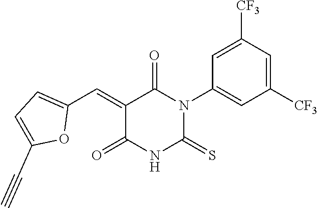 [SMILES: C#Cc1ccc(/C=C2\C(=O)NC(=S)N(c3cc(C(F)(F)F)cc(C(F)(F)F)c3)C2=O)o1]

Compound 5i
1-(3,5-
bis(trifluoromethyl)phenyl)-
5-((5-ethynylfuran-2-
yl)methylene)-2-
thioxodihydropyrimidine-
4,6(1H,5H)-dione

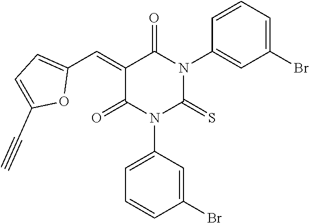 [SMILES: C#Cc1ccc(C=C2C(=O)N(c3cccc(Br)c3)C(=S)N(c3cccc(Br)c3)C2=O)o1]

Compound 5j
1,3-bis(3-bromophenyl)-5-
((5-ethynylfuran-2-
yl)methylene)-2-
thioxodihydropyrimidine-
4,6(1H,5H)-dione

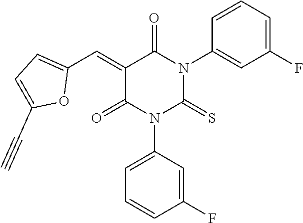 [SMILES: C#Cc1ccc(C=C2C(=O)N(c3cccc(F)c3)C(=S)N(c3cccc(F)c3)C2=O)o1]

Compound 5k
5-((5-ethynylfuran-2-yl)methylene)-1,3- bis(3-fluorophenyl)-2-
thioxodihydropyrimidine-4,6(1H,5H)-
dione

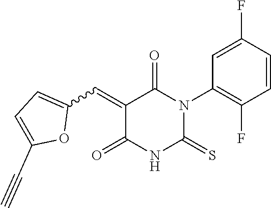 [SMILES: C#Cc1ccc(C=C2C(=O)NC(=S)N(c3cc(F)ccc3F)C2=O)o1]

Compound 5e
1-(2,5-difluorophenyl)-5-
((5-ethynylfuran-2-
yl)methylene)-2-
thioxodihydropyrimidine-
4,6(1H,5H)-dione

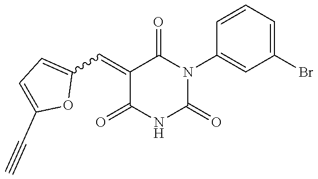 [SMILES: C#Cc1ccc(C=C2C(=O)NC(=O)N(c3cccc(Br)c3)C2=O)o1]

Compound 5l
1-(3-bromophenyl)-5-((5-
ethynylfuran-2-
yl)methylene)pyrimidine-
2,4,6(1H,3H,5H)-trione

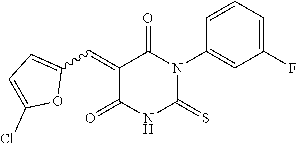 [SMILES: O=C1NC(=S)N(c2cccc(F)c2)C(=O)C1=Cc1ccc(Cl)o1]

Compound 6b
5-((5-chlorofuran-2-yl)methylene)-1-(3-
fluorophenyl)-2-thioxodihydropyrimidine-
4,6(1H,5H)-dione

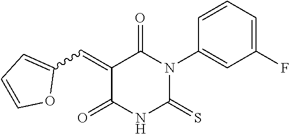 [SMILES: O=C1NC(=S)N(c2cccc(F)c2)C(=O)C1=Cc1ccco1]

Compound 6m
1-(3-fluorophenyl)-5-
(furan-2-ylmethylene)-2-
thioxodihydropyrimidine-
4,6(1H,5H)-dione

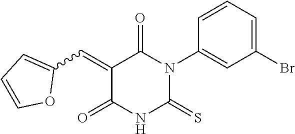 [SMILES: O=C1NC(=S)N(c2cccc(Br)c2)C(=O)C1=Cc1ccco1]

SMIFH2
1-(3-Bromophenyl)-5-(2-
furylmethylene)-2-thioxo-
hexahydropyrimidine-4,6-
dione

Optionally, a compound as disclosed in Table 1 can be used as inhibitor of interferon-γ mediated signaling. In particular, these compounds having an activity of inhibitor of interferon-γ mediated signaling can be a research tool, for instance as actin polymerization inhibitor. The present invention also relates to an in vitro or ex vivo method for inhibiting interferon-γ mediated signaling comprising contacting a sample comprising cells with a compound of Table 1, thereby inhibiting interferon-γ mediated signaling

### New Compounds

The invention also relates to a compound of general formula (I), or a pharmaceutically acceptable salt, stereoisomer, tautomer or solvate thereof, wherein,
X is an oxygen or sulfur atom;
R₁ is a chlorine atom or a (C₂-C₆)alkynyl group;
R₂ and R₃ are, independently of one another, a hydrogen atom, a (C₀-C₆)alkyl-ethynyl (—(CH₂)₀₋₆—C≡CH); a (C₀-C₃)alkyl-NH—C(O)—R′; a (C₀-C₃)alkyl-C(O)—NR′R″; a (C₀-C₃)alkyl-NH—C(O)—OR′; a (C₀-C₃)alkyl-NH—C(O)—NR′R″; a (C₀-C₃)alkyl-C(O)—R′; a (C₀-C₃)alkyl-C(O)—OR′; a (C₀-C₃)alkyl-NR′R″; a (C₀-C₃)alkyl-SOR′; a (C₀-C₃)alkyl-SO₂R′; a (C₀-C₃)alkyl-SONR′R″; a (C₀-C₃)alkyl-SO₂NR′R″; (C₀-C₃)alkyl-NHSO₂R′; or a group selected from (C₁-C₅)alkyl group, (C₁-C₅)alkyloxy group, (C₂-C₆)alkenyl, (C₂-C₆)alkynyl, (C₃-C₁₀)cycloalkyl group, (C₃-C₁₀)heterocycloalkyl group, (C₆-C₁₂)aryl group, or (C₅-C₁₂)heteroaryl group; said group being optionally substituted by at least one R;
R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group; and
R′ and R″ are independently selected from the group consisting of an hydrogen atom or a (C₁-C₆)alkyl group optionally substituted by at least one halogen;
provided that said compound is not a compound (Ia) or (Ib):

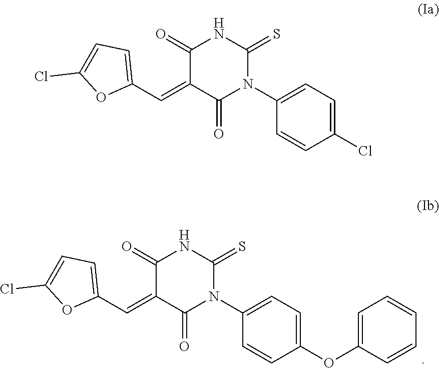 [SMILES: O=C1NC(=S)N(c2ccc(Cl)cc2)C(=O)/C1=C/c1ccc(Cl)o1 ; O=C1NC(=S)N(c2ccc(Oc3ccccc3)cc2)C(=O)/C1=C/c1ccc(Cl)o1]

In a specific aspect, the invention relates to the compound of general formula (I) in which R₁ is a chlorine or a (C₀-C₄)alkyl-ethynyl (—(CH₂)₀₋₄—C≡CH) such as an ethynyl, 2-propynyl or 3-butynyl. In a very specific aspect of the invention, R₁ is a chlorine or an ethynyl. In an even more specific aspect of the invention, R₁ is an ethynyl.

In a specific aspect, the invention relates to the compound of general formula (I) as defined above, in which X is a sulfur atom.

In a specific aspect, the invention relates to the compound of general formula (I) as defined above, in which X is a sulfur atom and R₁ is a chlorine or a (C₀-C₄)alkyl-ethynyl (—(CH₂)₀₋₄—C≡CH), preferably an ethynyl, 2-propynyl or 3-butynyl. In a very specific aspect of the invention, X is a sulfur atom and R₁ is a chlorine or an ethynyl. In an even more specific aspect of the invention, X is a sulfur atom and R₁ is an ethynyl.

In a specific aspect, the invention relates to the compound of general formula (I) as defined above, in which R₂ and R₃ are, independently of one another, a hydrogen atom or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)cycloheteroalkyl group, (C₆-C₁₂)aryl group, and (C₅-C₁₂)heteroaryl group; said group being optionally substituted by at least one R; provided that R₃ is not a hydrogen atom. In a more specific aspect, R₂ and R₃ are, independently of one another, a hydrogen atom or a group selected from (C₆-C₁₂)aryl group and (C₅-C₁₂)heteroaryl group; said group being optionally substituted by at least one R; provided that R₃ is not a hydrogen atom. In a more specific aspect, R₂ is H or a (C₆-C₁₂)aryl group optionally substituted by at least one R; and R₃ is a (C₆-C₁₂)aryl group optionally substituted by at least one R. In another more specific aspect, R₂ is H or a phenyl optionally substituted by at least one R; and R₃ is a phenyl optionally substituted by at least one R. Preferably, R₂ is H or a (C₆-C₁₂)aryl group substituted by at least one R; and R₃ is a (C₆-C₁₂)aryl group substituted by at least one R. More preferably, R₂ is H or a phenyl substituted by at least one R; and R₃ is a phenyl substituted by at least one R. More preferably, R₂ and R₃ are, independently of one another, a (C₆-C₁₂)aryl group substituted by at least one R, in particular by one or two R. Still more preferably, R₂ and R₃ are, independently of one another, a phenyl substituted by at least one R, in particular by one or two R. In a particular aspect, R₃ is preferably a phenyl group substituted by at least one R, in particular by one or two R. More particularly, R₃ is preferably a phenyl group substituted by at least one R, in particular by one or two R, and R₂ is and hydrogen atom or is identical to R₃.

In a specific aspect, the invention relates to the compound of general formula (I) as defined above, in which R₂ and R₃ are identical and are as defined above.

In a specific aspect, the invention relates to the compound of general formula (I) as defined above, in which:
X is a sulfur atom;
R₁ is a chlorine or a (C₀-C₄)alkyl-ethynyl (—(CH₂)₀₋₄—C≡CH) such as an ethynyl, 2-propynyl or 3-butynyl; and
R₂ and R₃ are, independently of one another, a hydrogen atom or a group selected from (C₆-C₁₂)aryl group and (C₅-C₁₂)heteroaryl group; said group being optionally substituted by at least one R;
provided that R₃ is not a hydrogen atom.

In a specific aspect, the invention relates to the compound of general formula (I) as defined above, in which:
X is a sulfur atom;
R₁ is a chlorine or an ethynyl;
R₂ is H or a (C₆-C₁₂)aryl group, preferably a phenyl, optionally substituted by at least one R; and
R₃ is a (C₆-C₁₂)aryl group, preferably a phenyl, optionally substituted by at least one R.

In a more specific aspect, the invention relates to the compound of general formula (I) as defined above, in which:
X is a sulfur atom;
R₁ is a chlorine or an ethynyl;
R₂ is H or a (C₆-C₁₂)aryl group, preferably a phenyl, substituted by at least one R; and
R₃ is a (C₆-C₁₂)aryl group, preferably a phenyl, substituted by at least one R.

In this aspect, R₂ and R₃ are preferably identical and are both a (C₆-C₁₂)aryl group, preferably a phenyl, substituted by at least one R. More preferably, R₂ and R₃ are identical and are both a phenyl group substituted by one or two R.

In a still more specific aspect, the invention relates to the compound of general formula (I) for use as defined above, in which:
X is a sulfur atom;
R₁ is an ethynyl;
R₂ is H or a (C₆-C₁₂)aryl group, preferably a phenyl, substituted by at least one R; and
R₃ is a (C₆-C₁₂)aryl group, preferably a phenyl, substituted by at least one R.

In another still more specific aspect, the invention relates to the compound of general formula (I) for use as defined above, in which:
X is a sulfur atom;
R₁ is a chlorine;
R₂ is H or a (C₆-C₁₂)aryl group, preferably a phenyl, substituted by at least one R; and
R₃ is a (C₆-C₁₂)aryl group, preferably a phenyl, substituted by at least one R selected from the group consisting of fluorine and —CF₃, preferably fluorine.

In the definitions of R₂ and R₃, R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group. In a specific aspect, R is independently selected from the group consisting of a halo selected in the group consisting of fluorine, bromine and chlorine; —CF₃; or a (C₁-C₆)alkyloxy group. In a more specific aspect, R is independently selected from the group consisting of fluorine, bromine, —CF₃, or a (C₁-C₃)alkyloxy group. Preferably, R is independently selected from the group consisting of fluorine, bromine, —CF₃, or a methoxy group (—OCH₃).

In the definitions, when R₂ and/or R₃ are a group substituted by more than one R, the R may be the same or different, preferably the R are identical.

In a very specific aspect, R₃ is a phenyl substituted by:
one substituent selected from the group consisting of bromine, fluorine, and —CF₃, in position ortho, meta or para, preferably ortho or meta;
two substituents selected from the group consisting of bromine, fluorine, and —CF₃, especially two fluorines or two —CF₃ groups, for instance either both in position meta or one in position ortho and the other in position meta.

In a very specific aspect, R₂ is H or a phenyl optionally substituted by:
one substituent selected from the group consisting of bromine, fluorine, and —CF₃, in position ortho, meta or para, preferably ortho or meta;
two substituents selected from the group consisting of bromine, fluorine, and —CF₃, especially two fluorines or two —CF₃ groups, for instance either both in position meta or one in position ortho and the other in position meta.

In an advantageous aspect of the invention, the compound of the invention is selected from the group consisting of compounds 5a, 5b, 5c, 5d, 5e, 5f, 5g, 5h, 5i, 5j, 5k, 5l, 6b, and 6c, as defined in Table 2.

TABLE 2

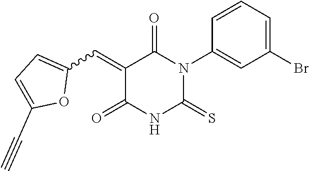 [SMILES: C#Cc1ccc(C=C2C(=O)NC(=S)N(c3cccc(Br)c3)C2=O)o1]

Compound 5a
(1-(3-bromophenyl)-5-((5-ethynylfuran-2-
yl)methylene)-2-thioxodihydropyrimidine-
4,6(1H,5H)-dione)

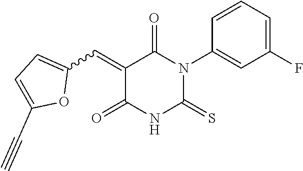 [SMILES: C#Cc1ccc(C=C2C(=O)NC(=S)N(c3cccc(F)c3)C2=O)o1]

Compound 5b
5-((5-ethynylfuran-2-
yl)methylene)-1-(3-
fluorophenyl)-2-
thioxodihydropyrimidine-
4,6(1H,5H)-dione

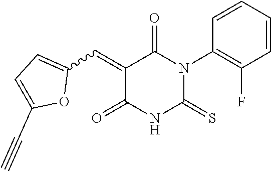 [SMILES: C#Cc1ccc(C=C2C(=O)NC(=S)N(c3ccccc3F)C2=O)o1]

Compound 5c
5-((5-ethynylfuran-2-
yl)methylene)-1-(2-
fluorophenyl)-2-
thioxodihydropyrimidine-
4,6(1H,5H)-dione

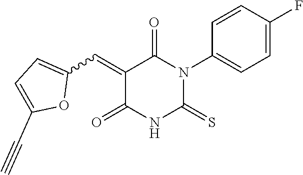 [SMILES: C#Cc1ccc(C=C2C(=O)NC(=S)N(c3ccc(F)cc3)C2=O)o1]

Compound 5d
5-((5-ethynylfuran-2-yl)methylene)-1-(4-
fluorophenyl)-2-thioxodihydropyrimidine-
4,6(1H,5H)-dione

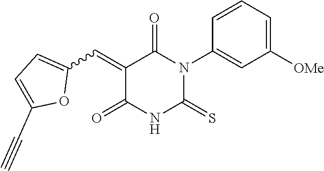 [SMILES: C#Cc1ccc(C=C2C(=O)NC(=S)N(c3cccc(OC)c3)C2=O)o1]

Compound 5f
5-((5-ethynylfuran-2-
yl)methylene)-1-(3-
methoxyphenyl)-2-
thioxodihydropyrimidine-
4,6(1H,5H)-dione

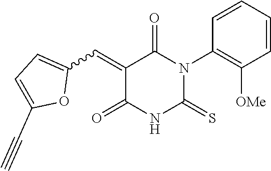 [SMILES: C#Cc1ccc(C=C2C(=O)NC(=S)N(c3ccccc3OC)C2=O)o1]

Compound 5g
5-((5-ethynylfuran-2-
yl)methylene)-1-(2-
methoxyphenyl)-2-
thioxodihydropyrimidine-
4,6(1H,5H)-dione

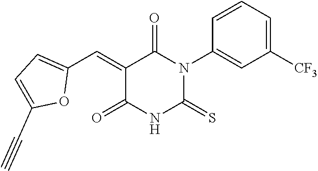 [SMILES: C#Cc1ccc(/C=C2\C(=O)NC(=S)N(c3cccc(C(F)(F)F)c3)C2=O)o1]

Compound 5h
5-((5-ethynylfuran-2-yl)methylene)-2-
thioxo-1-(3-
(trifluoromethyl)phenyl)dihydropyrimidine-
4,6(1H,5H)-dione

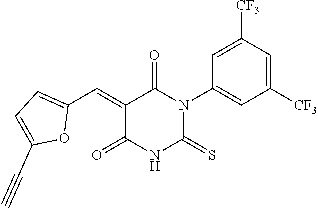 [SMILES: C#Cc1ccc(/C=C2\C(=O)NC(=S)N(c3cc(C(F)(F)F)cc(C(F)(F)F)c3)C2=O)o1]

Compound 5i
1-(3,5-
bis(trifluoromethyl)phenyl)-
5-((5-ethynylfuran-2-
yl)methylene)-2-
thioxodihydropyrimidine-
4,6(1H,5H)-dione

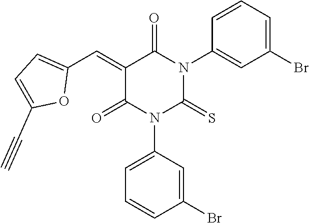 [SMILES: C#Cc1ccc(C=C2C(=O)N(c3cccc(Br)c3)C(=S)N(c3cccc(Br)c3)C2=O)o1]

Compound 5j
1,3-bis(3-bromophenyl)-5-
((5-ethynylfuran-2-
yl)methylene)-2-
thioxodihydropyrimidine-
4,6(1H,5H)-dione

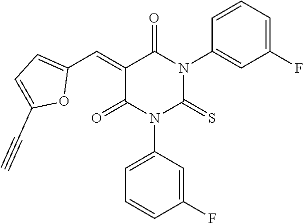 [SMILES: C#Cc1ccc(C=C2C(=O)N(c3cccc(F)c3)C(=S)N(c3cccc(F)c3)C2=O)o1]

Compound 5k
5-((5-ethynylfuran-2-yl)methylene)-1,3-
bis(3-fluorophenyl)-2-
thioxodihydropyrimidine-4,6(1H,5H)-
dione

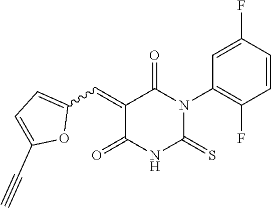 [SMILES: C#Cc1ccc(C=C2C(=O)NC(=S)N(c3cc(F)ccc3F)C2=O)o1]

Compound 5e
1-(2,5-difluorophenyl)-5-((5-
ethynylfuran-2-
yl)methylene)-2-
thioxodihydropyrimidine-
4,6(1H,5H)-dione

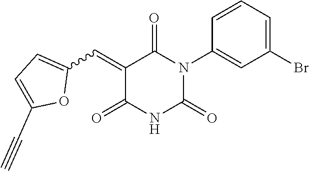 [SMILES: C#Cc1ccc(C=C2C(=O)NC(=O)N(c3cccc(Br)c3)C2=O)o1]

Compound 5l
1-(3-bromophenyl)-5-((5-
ethynylfuran-2-
yl)methylene)pyrimidine-
2,4,6(1H,3H,5H)-trione

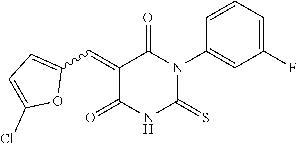 [SMILES: O=C1NC(=S)N(c2cccc(F)c2)C(=O)C1=Cc1ccc(Cl)o1]

Compound 6b
5-((5-chlorofuran-2-yl)methylene)-1-(3-
fluorophenyl)-2-thioxodihydropyrimidine-
4,6(1H,5H)-dione

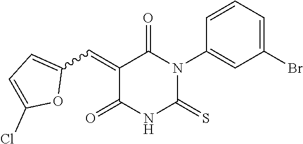 [SMILES: O=C1NC(=S)N(c2cccc(Br)c2)C(=O)C1=Cc1ccc(Cl)o1]

Compound 6c
5-((5-chlorofuran-2-
yl)methylene)-1-(3-
bromophenyl)-2-
thioxodihydropyrimidine-
4,6(1H,5H)-dione

The invention also relates to a compound of general formula (I) as defined above, especially one of those as disclosed in Table 2, or a pharmaceutical composition comprising it, for use as a drug.

The invention also relates to the use of a compound of general formula (I) as defined above, especially one of those as disclosed in Table 1, or a pharmaceutical composition comprising it, for the manufacture of a medicament.

The invention further relates to a method for treating a disease or disorder in a subject in need thereof, comprising administering a therapeutically effective amount of a compound of general formula (I) as defined above, especially one of those as disclosed in Table 1, to said subject.

### Therapeutic Uses

The invention relates to a compound of general formula (I) as defined above, especially one of those as disclosed in Table 1, or a pharmaceutical composition comprising it for use as inhibitor of interferon-γ mediated signaling. It further relates to the use of a compound of general formula (I) as defined above, especially one of those as disclosed in Table 1, or a pharmaceutical composition comprising it for the manufacture of a medicine for use as inhibitor of interferon-γ mediated signaling. It also relates to a method for inhibiting interferon-γ mediated signaling in a subject in need thereof, comprising administering a therapeutically effective amount of a compound of general formula (I) as defined above, especially one of those as disclosed in Table 1, or a pharmaceutical composition comprising it to said subject, thereby inhibiting interferon-γ mediated signaling.

More specifically, the invention relates to a compound of general formula (I) as defined above, especially one of those as disclosed in Table 1, or a pharmaceutical composition comprising it for use for preventing and/or treating diseases associated to the hyper-activation of interferon-γ mediated JAK/STAT signaling. It further relates to the use of a compound of general formula (I) as defined above, especially one of those as disclosed in Table 1, or a pharmaceutical composition comprising it for the manufacture of a medicine for use for preventing and/or treating diseases associated to the hyper-activation of interferon-γ mediated JAK/STAT signaling. It also relates to a method for preventing and/or treating diseases associated to the hyper-activation of interferon-γ mediated JAK/STAT signaling in a subject in need thereof, comprising administering a therapeutically effective amount of a compound of general formula (I) as defined above, especially one of those as disclosed in Table 1, or a pharmaceutical composition comprising it to said subject, thereby inhibiting interferon-γ mediated signaling.

In a specific aspect, the invention relates to a compound of general formula (I) as defined above, especially one of those as disclosed in Table 1, or a pharmaceutical composition comprising it for use for preventing and/or treating autoimmune and inflammation-associated diseases, viral diseases, atherosclerosis, metabolic syndrome, or cancer. It further relates to the use of a compound of general formula (I) as defined above, especially one of those as disclosed in Table 1, or a pharmaceutical composition comprising it for the manufacture of a medicine for use for preventing and/or treating autoimmune and inflammation-associated diseases, viral diseases, atherosclerosis, metabolic syndrome, or cancer. It also relates to a method for preventing and/or treating autoimmune and inflammation-associated diseases, viral diseases, atherosclerosis, metabolic syndrome, or cancer in a subject in need thereof, comprising administering a therapeutically effective amount of a compound of general formula (I) as defined above, especially one of those as disclosed in Table 1, or a pharmaceutical composition comprising it to said subject.

In particular, the invention relates to a compound of general formula (I) as defined above, especially one of those as disclosed in Table 1, or a pharmaceutical composition comprising it for use for preventing and/or treating a disease selected from the group consisting of Haemophagocytic Lymphohistiocytosis, Crohn's disease, Systemic Lupus Erythematosus, psoriasis, rheumatoid arthritis, ulcerative colitis, and coronavirus diseases. It further relates to the use of a compound of general formula (I) as defined above, especially one of those as disclosed in Table 1, or a pharmaceutical composition comprising it for the manufacture of a medicine for use for preventing and/or treating a disease selected from the group consisting of Haemophagocytic Lymphohistiocytosis, Crohn's disease, Systemic Lupus Erythematosus, psoriasis, rheumatoid arthritis, ulcerative colitis, and coronavirus diseases. It also relates to a method for preventing and/or treating a disease selected from the group consisting of Haemophagocytic Lymphohistiocytosis, Crohn's disease, Systemic Lupus Erythematosus, psoriasis, rheumatoid arthritis, ulcerative colitis, and coronavirus diseasesin a subject in need thereof, comprising administering a therapeutically effective amount of a compound of general formula (I) as defined above, especially one of those as disclosed in Table 1, or a pharmaceutical composition comprising it to said subject.

### Inflammation-Associated Diseases

Accordingly, the present invention relates to a compound of general formula (I) as disclosed herein, especially one of those as disclosed in Table 1, or a pharmaceutical composition comprising it for use for treating and/or preventing inflammation-associated diseases, and to the use of a compound general formula (I) as disclosed herein, especially one of those as disclosed in Table 1, or a pharmaceutical composition comprising it for the manufacture of a medicament useful for treating and/or preventing inflammation-associated diseases. It further relates to the a method for treating and/or preventing a subject suffering of inflammation-associated diseases, comprising administering a therapeutic effective amount of a compound of general formula (I) as disclosed herein, especially one of those as disclosed in Table 1, or a pharmaceutical composition comprising it to said subject.

The inflammation-associated diseases can be selected from the group consisting of a systemic inflammatory response syndrome, a cytokine release syndrome (CRS), an Adult Respiratory Distress Syndrome (ARDS), a Macrophage Activation Syndrome (MAS), an Alveolar inflammatory response, a paediatric multisystem inflammatory syndrome, a Hemophagocytic lymphohistiocytosis (HLH), systemic lupus erythematosus, a sepsis, in particular septic shock, Crohn's disease, ulcerative colitis, rheumatoid arthritis, inflammatory bowel disease or a hypercytokinemia. In particular, the inflammation-associated diseases can be selected from the group consisting of Haemophagocytic Lymphohistiocytosis, Crohn's disease, Systemic Lupus Erythematosus, rheumatoid arthritis and ulcerative colitis.

### Autoimmune Diseases or Disorders

In addition, the present invention also relates to a compound of general formula (I) as disclosed herein, especially one of those as disclosed in Table 1, or a pharmaceutical composition comprising it for use as anti-inflammatory agent or for use for treating and/or preventing an autoimmune disease or disorder, and to the use of a compound of general formula (I) as disclosed herein, especially one of those as disclosed in Table 1, or a pharmaceutical composition comprising it for the manufacture of a medicament for treating and/or preventing an autoimmune disease or disorder. It further relates to the treatment or prevention of a subject suffering of an autoimmune disease or disorder, comprising administering a therapeutic effective amount of a compound of general formula (I) as disclosed herein, especially one of those as disclosed in Table 1, or a pharmaceutical composition comprising it to said subject, thereby inducing an anti-inflammatory effect. Indeed, the current strategy for treating autoimmune diseases or disorders is to reduce inflammation.

The autoimmune disease or disorder can be selected from the group consisting of Addison disease, Hemolytic Autoimmune Anemia, Anti-Glomerular Basement Membrane Disease, Anti-Neutrophil Cytoplasmic Antibody-Associated Vasculitis including Churg-Strauss Syndrome, Granulomatosis with Polyangiitis and Microscopic Polyangiitis, Antiphospholipid Syndrome, Juvenile Arthritis, Rheumatoid Arthritis including Felty Syndrome, Rheumatoid Vasculitis, Sjogren's Syndrome and Adult-Onset Still's Disease, Autoimmune Diseases of the Nervous System including Anti-N-Methyl-D-Aspartate Receptor Encephalitis, Demyelinating Autoimmune Diseases, Myasthenia Gravis, Nervous System Autoimmune Disease, Polyradiculoneuropathy, Stiff-Person Syndrome, Uveomeningoencephalitic Syndrome, and CNS Vasculitis, Autoimmune Hypophysitis, Autoimmune Lymphoproliferative Syndrome, Autoimmune Pancreatitis, Birdshot Chorioretinopathy, Dermatitis Herpetiformis, Type 1 Diabetes Mellitus, Glomerulonephritis, Graves' Disease including Graves Ophthalmopathy, Autoimmune Hepatitis, Immunoglobulin G4-Related Disease, Latent Autoimmune Diabetes in Adults, Linear IgA Bullous Dermatosis, Systemic Lupus Erythematosus including Lupus Nephritis and Central Nervous System (CNS) Lupus Vasculitis, Sympathetic Ophthalmia, Bullous Pemphigoid, Pemphigus, Autoimmune Polyendocrinopathies, Idiopathic Thrombocytopenia Purpura, psoriasis and Autoimmune Thyroiditis.

In particular, the autoimmune disease or disorder can be selected from the group consisting of Systemic Lupus Erythematosus, psoriasis, or rheumatoid arthritis.

### Viral Diseases

Accordingly, the present invention relates to compounds of general formula (I) as disclosed herein, especially one of those as disclosed in Table 1, or a pharmaceutical composition comprising it, for use for treating and/or preventing viral diseases, and to the use of a compound of general formula (I) as disclosed herein, especially one of those as disclosed in Table 1, or a pharmaceutical composition comprising it for the manufacture of a medicament useful for the treatment and/or the prevention of a viral disease. It further relates to the method for treating and/or preventing a subject suffering of a viral disease, comprising administering a therapeutic effective amount of a compound as disclosed herein or a pharmaceutical composition comprising it to said subject.

The viral diseases can be for instance selected from the group consisting of respiratory viral diseases, hemorrhagic viral diseases, diseases caused by Epstein-Barr virus (EBV) and cytomegalovirus (CMV) and Arenaviruses such as Lassa virus (Remy et al. Cell Host Microbe 2017, PMID: 28826838). In particular, the viral diseases can be coronavirus diseases, such as a disease due to infection by Middle East respiratory syndrome-related coronavirus (MERS-COV), β-CoV, Severe acute respiratory syndrome coronavirus (SARS-CoV), β-CoV or Severe acute respiratory syndrome coronavirus 2 (SARS-COV-2), in particular COVID-19 or Severe COVID-19.

As used herein, the term “COVID-19” or “Coronavirus disease 2019” has its general meaning in the art and refers to an infectious coronavirus disease caused by severe acute respiratory syndrome coronavirus 2 (SARS-COV-2), a newly identified coronavirus in December 2019 in Wuhan, China. The term “COVID-19” also refers to 2019-nCOV acute respiratory disease. COVID-19 results in mild to moderate respiratory disease, but may in some cases develop into severe COVID-19.

As used herein, the term “Severe COVID-19” has its general meaning in the art and refers to COVID-19 side effect resulting in severe respiratory disease, pneumonia, viral sepsis, Cytokine Release Syndrome (CRS), Acute Respiratory Distress Syndrome (ARDS), Macrophage Activation Syndrome (MAS), multi-visceral failure syndrome caused by an enhanced inflammatory response such as kidney and lung failure, respiratory failure, arterial inflammation, myocarditis (also known as inflammatory cardiomyopathy), myocardial injury, thrombosis, venous thromboembolic event, cardiovascular diseases such as described in Han Y, Zeng H, Jiang H, Yang Y, Yuan Z, Cheng X, Jing Z, Liu B, Chen J, Nie S, Zhu J, Li F, Ma C. CSC Expert Consensus on Principles of Clinical Management of Patients with Severe Emergent Cardiovascular Diseases during the COVID-19 Epidemic. Circulation. 2020 Mar. 27. doi: 10.1161/CIRCULATIONAHA.120.047011), pulmonary embolism, neurologic toxicities, Kawasaki disease (also known as mucocutaneous lymph node syndrome) and Cutaneous manifestations of COVID-19 such as described in (Sachdeva M, Gianotti R, Shah M, Lucia B, Tosi D, Veraldi S, Ziv M, Leshem E, Dodiuk-Gad R P. Cutaneous manifestations of COVID-19: Report of three cases and a review of literature. J Dermatol Sci. 2020 Apr. 29. pii: S0923-1811 (20) 30149-3. doi: 10.1016/j.jdermsci.2020.04.011).

### Atherosclerosis

Accordingly, the present invention relates to compounds of general formula (I) as disclosed herein, especially one of those as disclosed in Table 1, or a pharmaceutical composition comprising it, for use for treating and/or preventing atherosclerosis, and to the use of a compound of general formula (I) as disclosed herein, especially one of those as disclosed in Table 1, or a pharmaceutical composition comprising it for the manufacture of a medicament useful for the treatment and/or the prevention of a atherosclerosis. It further relates to the method for treating and/or preventing a subject suffering of a atherosclerosis, comprising administering a therapeutic effective amount of a compound of general formula (I) as disclosed herein, especially one of those as disclosed in Table 1, or a pharmaceutical composition comprising it to said subject.

### Metabolic Diseases

Accordingly, the present invention relates to compounds of general formula (I) as disclosed herein, especially one of those as disclosed in Table 1, or a pharmaceutical composition comprising it, for use for treating and/or preventing metabolic diseases, and to the use of a compound of general formula (I) as disclosed herein, especially one of those as disclosed in Table 1, or a pharmaceutical composition comprising it for the manufacture of a medicament useful for the treatment and/or the prevention of a metabolic disease. It further relates to the method for treating and/or preventing a subject suffering of a metabolic disease, comprising administering a therapeutic effective amount of a compound of general formula (I) as disclosed herein, especially one of those as disclosed in Table 1, or a pharmaceutical composition comprising it to said subject.

The metabolic disease can be for instance selected from the group consisting of diabetes mellitus including type 1 and type 2 diabetes mellitus, insulin resistance, hyperglycemia, hyperinsulinemia, metabolic syndrome, glucose intolerance, hypertension, NAFLD, NASH and obesity (Sesta et al. 2018 Immunity PMID: 29958802; Herder 2018 Nat Rev Endo PMID: 30087397; Li et al 2021 Mol Immun PMID: 33770523; Zhang et al 20114 J of Hepatoc PMID: 25048951).

### Cancer

Accordingly, the present invention relates to compounds of general formula (I) as disclosed herein, especially one of those as disclosed in Table 1, or a pharmaceutical composition comprising it, for use as anti-tumoral agent or for use for treating and/or preventing a cancer, and to the use of a compound of general formula (I) as disclosed herein, especially one of those as disclosed in Table 1, or a pharmaceutical composition comprising it for the manufacture of a medicament useful as anti-tumoral agent or for treating and/or preventing a cancer. It further relates to the method for treating a subject suffering of a cancer, comprising administering a therapeutic effective amount of a compound of general formula (I) as disclosed herein, especially one of those as disclosed in Table 1, or a pharmaceutical composition comprising it to said subject, thereby inducing an anti-tumoral effect.

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarninoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Karposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pinealoma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Karposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), ear, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilm's tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

Optionally, the cancer can be selected in the group consisting of rectal cancer, colorectal cancer, stomach cancer, head and neck cancer, thyroid cancer, cervical cancer, uterine cancer, breast cancer, in particular triple negative breast cancer, ovarian cancer, brain cancer, in particular glioblastoma and neuroblastoma, lung cancer, in particular small-cell lung cancer and non-small-cell lung cancer, skin cancer, bladder cancer, blood cancer, renal cancer, liver cancer, prostate cancer, multiple myeloma, pancreatic cancer and endometrial cancer.

The term “cancer” according to the invention preferably comprises bladder cancer, pancreas cancer, lung carcinoma, hepatocellular carcinoma, gastric adenocarcinoma, hepatoma, mammary adenocarcinoma, and melanoma.

### Pharmaceutical Compositions

The pharmaceutical compositions contemplated herein may include a pharmaceutically acceptable carrier in addition to the active ingredient(s). The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parental administration, the active compounds(s) may be formulated in a unit dosage form for injection in vehicles such as saline, dextrose solution, serum albumin and Ringer's solution.

The pharmaceutical composition can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations suitable for parental administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavoring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

The pharmaceutical or veterinary composition as disclosed herein may further comprise an additional active ingredient or drug.

### Non-Therapeutic Uses

Accordingly, the invention also relates to the non-therapeutic use of a compound of general formula (I) as disclosed in Table 3, in particular selected from the group consisting of compounds 5l, 6a, 6e, 6j, 6k, 6l and 6m, preferably 6a, 6i, 6j, 6k and 6l, as inhibitor of formin FH2 domains. It relates to the use of a compound of Table 3 for inhibiting actin polymerization, and/or for inhibiting formin-mediated actin nucleation and/or for inhibiting formin-mediated elongation of actin filaments.

TABLE 3

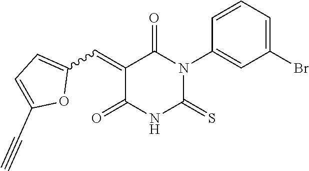 [SMILES: C#Cc1ccc(C=C2C(=O)NC(=S)N(c3cccc(Br)c3)C2=O)o1]

Compound 5l
1-(3-bromophenyl)-5-((5-
ethynylfuran-2-
yl)methylene)pyrimidine-
2,4,6(1H,3H,5H)-trione

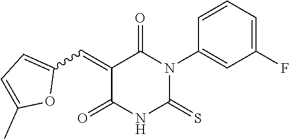 [SMILES: Cc1ccc(C=C2C(=O)NC(=S)N(c3cccc(F)c3)C2=O)o1]

Compound 6a
1-(3-fluorophenyl)-5-((5-
methylfuran-2-yl)methylene)-2-
thioxodihydropyrimidine-
4,6(1H,5H)-dione

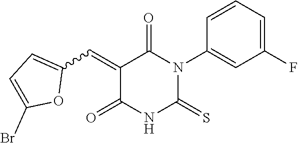 [SMILES: O=C1NC(=S)N(c2cccc(F)c2)C(=O)C1=Cc1ccc(Br)o1]

Compound 6i
5-((5-bromofuran-2-
yl)methylene)-1-(3-
fluorophenyl)-2-
thioxodihydropyrimidine-
4,6(1H,5H)-dione

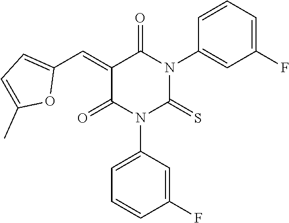 [SMILES: Cc1ccc(C=C2C(=O)N(c3cccc(F)c3)C(=S)N(c3cccc(F)c3)C2=O)o1]

Compound 6j
1,3-bis(3-fluorophenyl)-5-((5-
methylfuran-2-yl)methylene)-2-
thioxodihydropyrimidine-
4,6(1H,5H)-dione

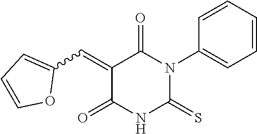 [SMILES: O=C1NC(=S)N(c2ccccc2)C(=O)C1=Cc1ccco1]

Compound 6k
5-(furan-2-ylmethylene)-1-
phenyl-2-
thioxodihydropyrimidine-
4,6(1H,5H)-dione

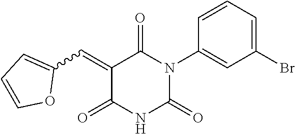 [SMILES: O=C1NC(=O)N(c2cccc(Br)c2)C(=O)C1=Cc1ccco1]

Compound 6l
1-(3-bromophenyl)-5-(furan-2-
ylmethylene)pyrimidine-
2,4,6(1H,3H,5H)-trione

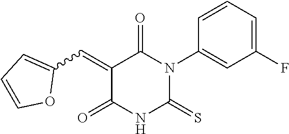 [SMILES: O=C1NC(=S)N(c2cccc(F)c2)C(=O)C1=Cc1ccco1]

Compound 6m
1-(3-fluorophenyl)-5-(furan-2-
ylmethylene)-2-
thioxodihydropyrimidine-
4,6(1H,5H)-dione

In particular, these compounds having an activity of inhibitor of formin FH2 domains can be a research tool, for instance as actin polymerization inhibitor. The present invention also relates to an in vitro or ex vivo method for inhibiting actin polymerization comprising contacting a sample comprising cells with a compound of Table 3, thereby inhibiting actin polymerization. It further relates to an in vitro or ex vivo method for inhibiting formin-mediated actin nucleation and/or formin-mediated elongation of actin filaments comprising contacting a sample comprising cells with a compound of Table 3, thereby inhibiting formin-mediated actin nucleation and/or formin-mediated elongation of actin filaments.

Further aspects and advantages of the present invention will be described in the following examples, which should be regarded as illustrative and not limiting.

### EXAMPLES

### Example 1: Synthesis of Compounds

Table 4 summarizes the compounds that have been prepared.

TABLE 4

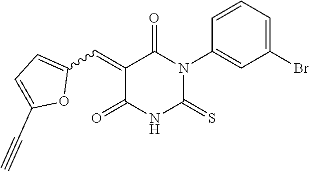 [SMILES: C#Cc1ccc(C=C2C(=O)NC(=S)N(c3cccc(Br)c3)C2=O)o1]

Compound 5a
(1-(3-bromophenyl)-5-((5-ethynylfuran-2-
yl)methylene)-2-
thioxodihydropyrimidine-4,6(1H,5H)-
dione)

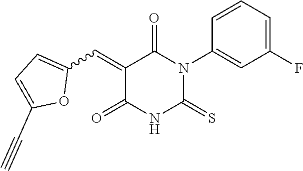 [SMILES: C#Cc1ccc(C=C2C(=O)NC(=S)N(c3cccc(F)c3)C2=O)o1]

Compound 5b
5-((5-ethynylfuran-2-
yl)methylene)-1-(3-
fluorophenyl)-2-
thioxodihydropyrimidine-
4,6(1H,5H)-dione

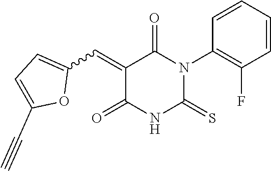 [SMILES: C#Cc1ccc(C=C2C(=O)NC(=S)N(c3ccccc3F)C2=O)o1]

Compound 5c
5-((5-ethynylfuran-2-
yl)methylene)-1-(2-
fluorophenyl)-2-
thioxodihydropyrimidine-
4,6(1H,5H)-dione

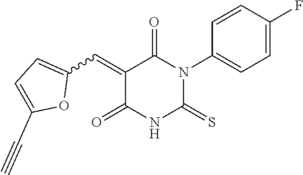 [SMILES: C#Cc1ccc(C=C2C(=O)NC(=S)N(c3ccc(F)cc3)C2=O)o1]

Compound 5d
5-((5-ethynylfuran-2-yl)methylene)-1-(4-
fluorophenyl)-2-thioxodihydropyrimidine-
4,6(1H,5H)-dione

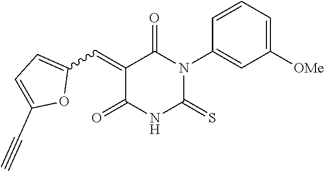 [SMILES: C#Cc1ccc(C=C2C(=O)NC(=S)N(c3cccc(OC)c3)C2=O)o1]

Compound 5f
5-((5-ethynylfuran-2-
yl)methylene)-1-(3-
methoxyphenyl)-2-
thioxodihydropyrimidine-
4,6(1H,5H)-dione

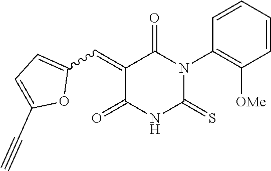 [SMILES: C#Cc1ccc(C=C2C(=O)NC(=S)N(c3ccccc3OC)C2=O)o1]

Compound 5g
5-((5-ethynylfuran-2-
yl)methylene)-1-(2-
methoxyphenyl)-2-
thioxodihydropyrimidine-
4,6(1H,5H)-dione

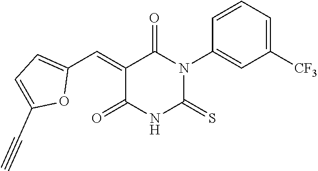 [SMILES: C#Cc1ccc(/C=C2\C(=O)NC(=S)N(c3cccc(C(F)(F)F)c3)C2=O)o1]

Compound 5h
5-((5-ethynylfuran-2-yl)methylene)-2-
thioxo-1-(3-
(trifluoromethyl)phenyl)dihydropyrimidine-
4,6(1H,5H)-dione

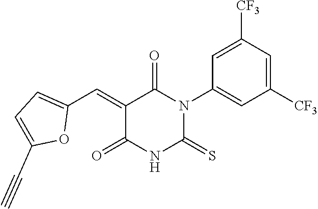 [SMILES: C#Cc1ccc(/C=C2\C(=O)NC(=S)N(c3cc(C(F)(F)F)cc(C(F)(F)F)c3)C2=O)o1]

Compound 5i
1-(3,5-
bis(trifluoromethyl)phenyl)-
5-((5-ethynylfuran-2-
yl)methylene)-2-
thioxodihydropyrimidine-
4,6(1H,5H)-dione

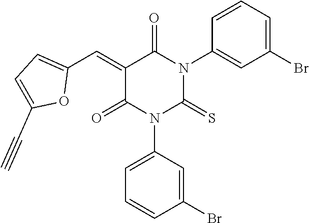 [SMILES: C#Cc1ccc(C=C2C(=O)N(c3cccc(Br)c3)C(=S)N(c3cccc(Br)c3)C2=O)o1]

Compound 5j
1,3-bis(3-bromophenyl)-5-
((5-ethynylfuran-2-
yl)methylene)-2-
thioxodihydropyrimidine-
4,6(1H,5H)-dione

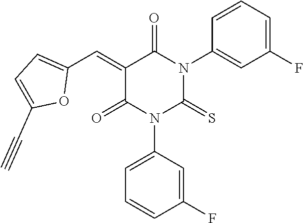 [SMILES: C#Cc1ccc(C=C2C(=O)N(c3cccc(F)c3)C(=S)N(c3cccc(F)c3)C2=O)o1]

Compound 5k
5-((5-ethynylfuran-2-yl)methylene)-1,3-
bis(3-fluorophenyl)-2-
thioxodihydropyrimidine-4,6(1H,5H)-
dione

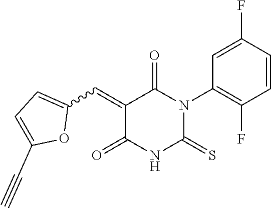 [SMILES: C#Cc1ccc(C=C2C(=O)NC(=S)N(c3cc(F)ccc3F)C2=O)o1]

Compound 5e
1-(2,5-difluorophenyl)-5-
((5-ethynylfuran-2-
yl)methylene)-2-
thioxodihydropyrimidine-
4,6(1H,5H)-dione

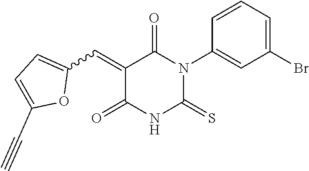 [SMILES: C#Cc1ccc(C=C2C(=O)NC(=S)N(c3cccc(Br)c3)C2=O)o1]

Compound 5l
1-(3-bromophenyl)-5-((5-
ethynylfuran-2-
yl)methylene)pyrimidine-
2,4,6(1H,3H,5H)-trione

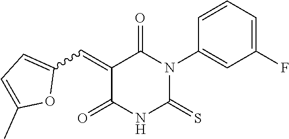 [SMILES: Cc1ccc(C=C2C(=O)NC(=S)N(c3cccc(F)c3)C2=O)o1]

Compound 6a
1-(3-fluorophenyl)-5-((5-methylfuran-2-
yl)methylene)-2-
thioxodihydropyrimidine-4,6(1H,5H)-
dione

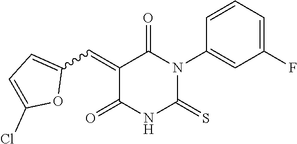 [SMILES: O=C1NC(=S)N(c2cccc(F)c2)C(=O)C1=Cc1ccc(Cl)o1]

Compound 6b
5-((5-chlorofuran-2-
yl)methylene)-1-(3-
fluorophenyl)-2-
thioxodihydropyrimidine-
4,6(1H,5H)-dione

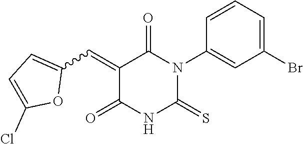 [SMILES: O=C1NC(=S)N(c2cccc(Br)c2)C(=O)C1=Cc1ccc(Cl)o1]

Compound 6c

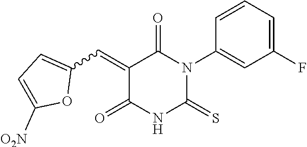 [SMILES: O=C1NC(=S)N(c2cccc(F)c2)C(=O)C1=Cc1ccc([N+](=O)[O-])o1]

Compound 6e
1-(3-fluorophenyl)-5-((5-nitrofuran-2-
yl)methylene)-2-
thioxodihydropyrimidine-4,6(1H,5H)-
dione

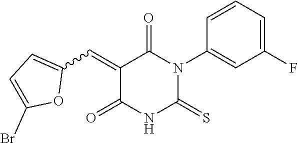 [SMILES: O=C1NC(=S)N(c2cccc(F)c2)C(=O)C1=Cc1ccc(Br)o1]

Compound 6i
5-((5-bromofuran-2-
yl)methylene)-1-(3-
fluorophenyl)-2-
thioxodihydropyrimidine-
4,6(1H,5H)-dione

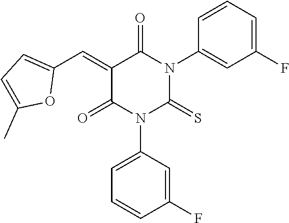 [SMILES: Cc1ccc(C=C2C(=O)N(c3cccc(F)c3)C(=S)N(c3cccc(F)c3)C2=O)o1]

Compound 6j
1,3-bis(3-fluorophenyl)-5-
((5-methylfuran-2-
yl)methylene)-2-
thioxodihydropyrimidine-
4,6(1H,5H)-dione

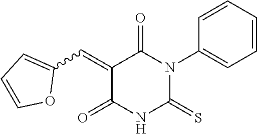 [SMILES: O=C1NC(=S)N(c2ccccc2)C(=O)C1=Cc1ccco1]

Compound 6k
5-(furan-2-ylmethylene)-1-phenyl-2-
thioxodihydropyrimidine-4,6(1H,5H)-
dione

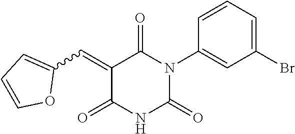 [SMILES: O=C1NC(=O)N(c2cccc(Br)c2)C(=O)C1=Cc1ccco1]

Compound 6l
1-(3-bromophenyl)-5-
(furan-2-
ylmethylene)pyrimidine-
2,4,6(1H,3H,5H)-trione

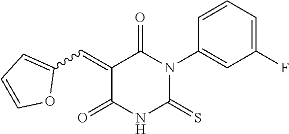 [SMILES: O=C1NC(=S)N(c2cccc(F)c2)C(=O)C1=Cc1ccco1]

Compound 6m
1-(3-fluorophenyl)-5-
(furan-2-ylmethylene)-2-
thioxodihydropyrimidine-
4,6(1H,5H)-dione

### General Information:

All solvents and chemicals were purchased from commercially available sources and used without further purification. Solvents were dried under standard conditions. Reactions were monitored by thin layer chromatography (TLC) using precoated silica on aluminum plates from Merck (60 F₂₅₄). TLC plates were visualized with UV light. Products were purified on column chromatography with Silica gel 60 from Alfa Aesar (0.036-0.071 mm; 215-400 mesh), a Combiflash RF+ Teledyne Isco system fitted with pre-packed silica gel columns (Interchim, 4-300 g columns, 50 μm particle size), a preparative HPLC Quaternary Gradient 2545 equipped with a Photodiode Array detector (Waters) fitted with a reverse phase column (XBridge Prep C₁₈ 5 μm OBD 30×150 mm).

NMR spectroscopy was performed on Bruker spectrometers. Spectra were run at 298 K unless stated otherwise. ¹H-NMR were recorded at 400 or 500 MHZ, and chemical shifts δ are expressed in ppm using the residual non-deuterated solvent signal as internal standard and the coupling constants J are specified in Hz and E/Z to denote the signal from isomers. The following abbreviations are used: s, singlet; d, doublet; dd, doublet of doublets; t, triplet; td, triplet of doublets; q, quadruplet; m, multiplet; bs, broad signal. We only reported labile protons that could be clearly identified in the spectra, namely for compounds 2c, 2f, 7a, 7b and 7c. ¹³C-NMR were recorded at 101 or 126 MHZ, and chemical shifts δ ppm are expressed in ppm using deuterated solvent signal as internal standard and the coupling constants J with Fluorine are specified in Hz. “/” is used in between the ppm signals to denote signal from E/Z isomers.

¹⁹F-NMR were recorded at 376 or 471 MHz, and chemical shifts δ are expressed in ppm. Molecular structures were characterized using a comprehensive dataset including ¹H- and ¹³C-NMR spectra (1D and 2D experiments including COSY, HMBC, and HSQC).

The purity of final compounds, determined to be >95% by UPLC-MS, and low-resolution mass spectra (LRMS) were recorded on a Waters Acquity H-class equipped with a Photodiode array detector and SQ Detector 2 fitted with a reverse phase column (Aquity UPLC® BEH C₁₈ 1.7 μm, 2.1×50 mm). High resolution mass spectra (HRMS) were recorded on a Thermo Fisher Scientific Q-Exactive Plus equipped with a Robotic TriVersa NanoMate Advion.

Abbreviations used. ACN, acetonitrile; AcOH, acetic acid; aq., aqueous; DCM, dichloromethane; DMSO, dimethylsulfoxide; eq, equivalent(s); cHex, cyclohexane; EtOAc, ethyl acetate; Et₃N, triethylamine; ESI, electrospray ionization; HPLC, high pressure liquid chromatography; HRMS, high resolution mass spectroscopy; LRMS, low resolution mass spectroscopy; MeOH, methanol; min, minutes; hrs, hours; MS, mass spectrometry; NMR, nuclear magnetic resonance; r.t., room temperature; TMS, trimethylsilane; THF, tetrahydrofuran; TLC, thin-layer chromatography.

### 1. Synthesis of 5-((trimethylsilyl) ethynyl) furan-2-carbaldehyde 1a

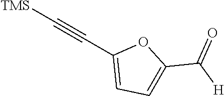 [SMILES: C[Si](C)(C)C#Cc1ccc(C=O)o1]

5-((trimethylsilyl) ethynyl) furan-2-carbaldehyde 1a was synthesized according to published procedure¹. Trimethylsilylacetylene (835 μL, 1.05 eq) and Et₃N (2 mL) were dissolved in THF (5 mL). The mixture was degassed three times and stirred under argon. 5-bromofuran-2-carbaldehyde (1.00 g, 5.71 mmoles) and PdCl₂(PPh₃)₂ (80 mg, 2 mol %) were added. After 1 min of stirring, CuI (43.5 mg, 4 mol %) was added, then the mixture was stirred at r.t. for 16 hrs then concentrated under reduced pressure. The crude product was purified by flash chromatography on silica gel (cHex/EtOAc, 6/4) to give of designed product, pale yellow crystals. Percentage yield: 626.5 mg, 57%.

¹H NMR (400 MHZ, CDCl₃) δ ppm: 9.61 (s, 1H), 7.19 (d, J=3.7 Hz, 1H), 6.71 (d, J=3.7 Hz, 1H), 0.27 (s, 9H). HRMS (ESI+) m/z: calculated for C₁₀H₁₃O₂Si⁺ (M+H)⁺: 193.0607; found: 193.0680

### 2. Synthesis of Thiobarbituric Acid Derivatives

Analogs of thiobarbituric acids were synthesised according to published procedures^{2,3}. To a suspension of thiourea derivative (1 eq) in propanol (5 ml), diethyl malonate (2.5 eq) and sodium methoxide (25% wt in MeOH or powder, 2.5 eq) were added under Argon atmosphere. The reaction mixture was stirred under reflux (105° C.) overnight. Completion of the reaction was checked by TLC analysis and UPLC-MS. The crude was then cooled to r.t., and quenched with acetic acid to reach about pH=6-7. The solvent was evaporated in vacuo, and the product was purified from the crude by flash chromatography on silica gel or using a CombiFlash system (DCM/MeOH, 8/2).

The Keto-Enol Tautomers are observed in the NMR spectra and their corresponding forms are stated in the description. In case of MeOD-d₄ as solvent, due to exchange with MeOD-d₄, the protons of the Keto-Enol cannot be observed which is concordant with the literature³.

### 1-(3-fluorophenyl)-2-thioxodihydropyrimidine-4,6(1H,5H)-dione 2a

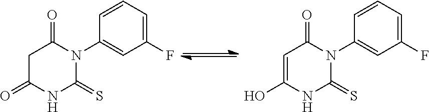 [SMILES: O=C1CC(=O)N(c2cccc(F)c2)C(=S)N1 ; O=c1cc(O)[nH]c(=S)n1-c1cccc(F)c1]

From 1-(3-fluorophenyl)thiourea (400 mg, 2.35 mmoles), Diethyl malonate (896 μL), sodium methoxide (25% solution in MeOH) (1343 μL).

Light Yellow powder, Percentage Yield=89.6%

¹H NMR (400 MHZ, DMSO-d₆) δ ppm: 10.66 (bs, 1H), 7.48-7.25 (m, 1H), 7.20-7.03 (m, 1H), 6.96-6.82 (m, 2H), 4.24 (s, 1H).Enol Form. ¹³C NMR (101 MHZ, DMSO-d₆) δ ppm: 176.53, 163.41, 162.37 (d, J_{C-F}=242.4 Hz,), 162.90, 143.40 (d, J_{C-F}=10.7 Hz), 129.80 (d, J_{C-F}=8.7 Hz), 126.56, 117.46 (d, J_{C-F}=22.1 Hz), 114.13 (d, J_{C-F}=20.9 Hz), 79.76. ¹⁹F NMR (376 MHZ, DMSO-d₆) δ ppm: −114.65. HRMS (ESI+) m/z: calculated for C₁₀H₈FN₂O₂S⁺ (M+H)⁺: 239.0212; found: 239.0285

### 1-(4-fluorophenyl)-2-thioxodihydropyrimidine-4,6(1H,5H)-dione 2b

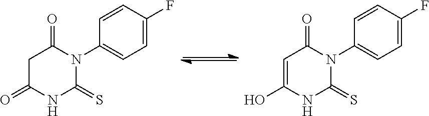 [SMILES: O=C1CC(=O)N(c2ccc(F)cc2)C(=S)N1 ; O=c1cc(O)[nH]c(=S)n1-c1ccc(F)cc1]

From 1-(4-fluorophenyl)thiourea (250 mg, 1.47 mmoles), Diethyl malonate (558 μL), sodium methoxide (25% solution in MeOH) (671 μL). Yellow powder, Percentage Yield=248 mg, 71%.

¹H NMR (500 MHZ, DMSO-d₆) δ ppm: 10.69 (bs, 1H), 7.16 (t, J_{C-F}=8.8 Hz, 2H), 7.05 (dd, J_{C-F}=8.7 Hz, J=5.0 Hz, 2H), 4.27 (s, 1H, 80% Enol), 3.17 (d, J=4.5 Hz, 1H, 15% Keto). ¹³C NMR (126 MHZ, DMSO-d₆) δ ppm: 176.81, 163.64, 162.99, 161.27 (d, J_{C-F}==242.1 Hz), 137.93, 131.90 (d, J_{C-F}=8.7 Hz, 2C), 115.30 (d, J_{C-F}=22.6 Hz, 2C), 79.90. ¹⁹F NMR (376 MHZ, DMSO-d₆) δ ppm: −116.23. HRMS (ESI+) m/z: calculated for C₁₀H₈FN₂O₂S⁺ (M+H)⁺: 239.0212; found: 239.0285

### 1-(2-fluorophenyl)-2-thioxodihydropyrimidine-4,6(1H,5H)-dione 2c

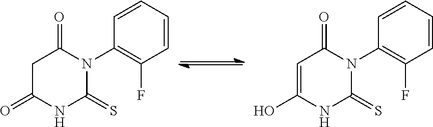 [SMILES: O=C1CC(=O)N(c2ccccc2F)C(=S)N1 ; O=c1cc(O)[nH]c(=S)n1-c1ccccc1F]

From 1-(4-fluorophenyl)thiourea (250 mg, 1.47 mmoles), Diethyl malonate (558 μL), sodium methoxide (25% solution in MeOH) (671 μL). Yellow powder, Percentage Yield=219 mg, 63%.

¹H NMR (500 MHZ, MeOD-d₄) δ ppm: 7.35-7.23 (m, 1H), 7.19-7.01 (m, 3H), 3.25 (s, 1H). ¹³C NMR (126 MHz, MeOD-d₄) δ ppm: 177.07, 165.47, 164.84, 158.24 (d, J_{C-F}=249.7 Hz), 131.00, 129.65 (d, J_{C-F}=7.9 Hz), 127.65 (d, J_{C-F}=13.4 Hz), 123.96, 115.55 (d, J_{C-F}=20.0 Hz), 48.46 (Keto form). ¹⁹F NMR (376 MHZ, MeOD-d₄) δ ppm: −123.64. HRMS (ESI+) m/z: calculated for C₁₀H₈FN₂O₂S⁺ (M+H)⁺: 239.0212; found: 239.0285

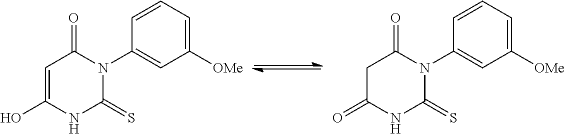 [SMILES: COc1cccc(-n2c(=O)cc(O)[nH]c2=S)c1 ; COc1cccc(N2C(=O)CC(=O)NC2=S)c1]

### 1-(3-methoxyphenyl)-2-thioxodihydropyrimidine-4,6(1H,5H)-dione) 2d

From 1-(3-methoxyphenyl)thiourea (250 mg, 1.37 mmoles), Diethyl malonate (525 μL), sodium methoxide (25% solution in MeOH) (630 μL). Pale white powder, Percentage Yield: 283 mg, 83%.

¹H NMR (400 MHZ, DMSO-d₆) δ ppm: 10.61 (bs, 1H), 7.25 (t, J=8.0 Hz, 1H), 6.84 (ddd, J=8.3, 2.5, 0.9 Hz, 1H), 6.64-6.51 (m, 2H), 4.26 (s, 1H, 50% Enol), 3.74 (s, 3H), 3.17 (s, 1H, 50% Keto). ¹³C NMR (101 MHZ, DMSO-d₆) δ ppm: 176.48, 163.61, 162.88, 159.67, 142.80, 129.12, 122.45, 115.93, 112.77, 79.86 (Enol), 55.56, 48.98 (Keto). HRMS (ESI+) m/z: calculated for C₁₁H₁₁N₂O₃S⁺ (M+H)⁺: 251.0412; found: 251.0485

### 1-(2-methoxyphenyl)-2-thioxodihydropyrimidine-4,6(1H,5H)-dione) 2e

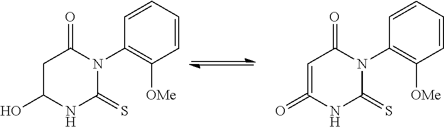

From 1-(2-methoxyphenyl)thiourea (250 mg, 1.37 mmoles), Diethyl malonate (525 μL), sodium methoxide (25% solution in MeOH) (630 μL). Pale white powder, Percentage Yield: 311 mg, 91%.

¹H NMR (400 MHZ, DMSO-d₆) δ ppm: 10.62 (bs, 1H), 7.25 (ddd, J=8.3, 6.9, 2.2 Hz, 1H), 7.00 (d, J=7.8 Hz, 1H), 6.97-6.87 (m, 2H), 4.24 (s, 1H, 68% Enol), 3.69 (s, 3H), 3.17 (s, 1H, 32% Keto). ¹³C NMR (101 MHZ, DMSO-d₆) δ ppm: 176.74, 163.37, 162.91, 155.52, 131.07, 130.36, 128.75, 120.37, 112.29, 79.91 (68% Enol), 55.88, 49.00 (32% Keto). HRMS (ESI+) m/z: calculated for C₁₁H₁₁N₂O₃S⁺ (M+H)⁺: 251.0412; found: 251.0485

### 1-(2,5-difluorophenyl)-2-thioxodihydropyrimidine-4,6(1H,5H)-dione 2f

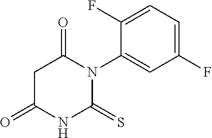 [SMILES: O=C1CC(=O)N(c2cc(F)ccc2F)C(=S)N1]

From 1-(2,5-difluorophenyl)thiourea (500 mg, 2.66 mmoles), Diethyl malonate (403 μL), sodium methoxide (25% solution in MeOH) (1518 μL). Light yellow powder, Percentage Yield: 572 mg, 84%.

¹H NMR (500 MHZ, MeOD-d₄) δ ppm: 7.06 (2H, ddd, J=15.1, 8.2, 4.0 Hz), 6.97-6.84 (1H, m), 3.25 (1H, s, exchanged with MeOD-d₄). ¹³C NMR (126 MHZ, MeOD-d₄) δ ppm: 176.90, 165.25, 165.04, 158.39 (d, J_{C-F}=241.1 Hz), 154.79 (d, J_{C-F}=244.4 Hz), 128.56 (dd, J_{C-F}=15.3, 11.6 Hz), 117.93 (d, J_{C-F}=25.4 Hz), 116.31 (dd, J_{C-F}=22.9, 9.4 Hz), 115.93 (dd, J_{C-F}=24.1, 8.1 Hz), 48.47. ¹⁹F NMR (376 MHZ, MeOD-d₄) δ ppm: −120.90 (d, J_{F-F}=16.0 Hz), −128.75 (d, J_{F-F}=16.1 Hz). HRMS (ESI+) m/z: calculated for C₁₀H₇F₂N₂O₂S⁺ (M+H)⁺: 257.0118; found: 257.0189

### 1-(3-bromophenyl)-2-thioxodihydropyrimidine-4,6(1H,5H)-dione 2g

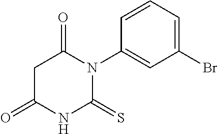 [SMILES: O=C1CC(=O)N(c2cccc(Br)c2)C(=S)N1]

From 1-(3-bromophenyl)thiourea (200 mg, 0.87 mmoles), Diethyl malonate (129 μL), sodium methoxide (25% solution in MeOH) (495 μL). Light pale yellow powder. Percentage Yield: 115 mg, 52%.

¹H NMR (400 MHZ, CD₃CN) δ ppm: 10.73 (bs, 1H), 7.53 (m, 1H), 7.40 (t, J=1.9 Hz, 1H), 7.35 (t, J=8.0 Hz, 1H), 7.20 (m, 1H), 3.30 (s, 2H), 1.99 (s, 1H, Acetic Acid). ¹³C NMR (101 MHZ, CD₃CN) δ ppm 177.66, 174.08 (Acetic Acid), 165.31, 165.05, 142.90, 133.06, 131.02, 130.71, 129.31, 121.53, 49.47, 20.69 (Acetic Acid). HRMS (ESI+) m/z: calculated for C₁₀H₈BrN₂O₂S⁺ (M+H)⁺: 298.9412; found: 300.9464.

### 2-thioxo-1-(3-(trifluoromethyl)phenyl)dihydropyrimidine-4,6(1H,5H)-dione 2h

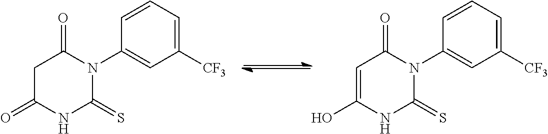 [SMILES: O=C1CC(=O)N(c2cccc(C(F)(F)F)c2)C(=S)N1 ; O=c1cc(O)[nH]c(=S)n1-c1cccc(C(F)(F)F)c1]

From 1-(3-(trifluoromethyl)phenyl)thiourea (200 mg, 0.91 mmoles), Diethyl malonate (207 μL), sodium methoxide (25% solution in MeOH) (520 μL). Light yellow powder. Percentage Yield: 259 mg, 99%.

¹H NMR (500 MHZ, DMSO-d₆) δ ppm: 10.80 (bs, 1H), 7.69-7.55 (m, 2H), 7.46-7.32 (m, 2H), 4.27 (s, 1H). ¹³C NMR (126 MHZ, DMSO-d₆) δ ppm: 176.63, 163.38, 162.90, 142.49, 134.59, 129.75, 129.43 (q, J_{C-F}=31.9 Hz), 126.98/126.94, 124.58 (q, J_{C-F}=272.0 Hz), 124.09, 79.80. ¹⁹F NMR (376 MHZ, DMSO-d₆) δ ppm: −60.84. HRMS (ESI+) m/z: calculated for C₁₁H₈F₃N₂O₂S⁺ (M+H)⁺: 289.0180; found: 289.0253.

### 1-(3,5-bis(trifluoromethyl)phenyl)-2-thioxodihydropyrimidine-4,6(1H,5H)-dione 2i

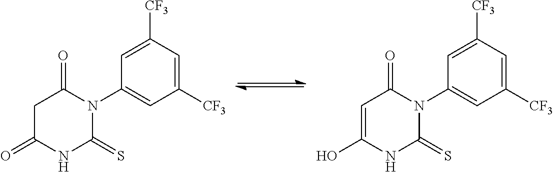 [SMILES: O=C1CC(=O)N(c2cc(C(F)(F)F)cc(C(F)(F)F)c2)C(=S)N1 ; O=c1cc(O)[nH]c(=S)n1-c1cc(C(F)(F)F)cc(C(F)(F)F)c1]

From 1-(3,5-bis(trifluoromethyl)phenyl)thiourea (200 mg, 0.69 mmoles), Diethyl malonate (116 μL), sodium methoxide (94 mg). Light yellow powder. Percentage Yield: 64 mg, 26%

¹H NMR (400 MHZ, DMSO-d₆) δ ppm: 11.02 (bs, 1H), 8.09 (s, 1H), 7.87 (d, J=1.5 Hz, 2H), 4.35 (s, 1H). ¹³C NMR (101 MHZ, DMSO-d₆) δ ppm: 176.50, 163.08, 162.86, 143.77, 131.68 (2C), 130.62 (d, J_{C-F}=33.0 Hz, 2C), 123.69 (d, J_{C-F}=272.8 Hz, 2C), 121.22, 79.79. ¹⁹F NMR (376 MHZ, DMSO-d₆) δ ppm: −61.12. HRMS (ESI+) m/z: calculated for C₁₂H₇F₆N₂O₂S⁺ (M+H)⁺: 357.0054; found: 357.0127.

### 1-(3-bromophenyl)pyrimidine-2,4,6(1H,3H,5H)-trione 2j

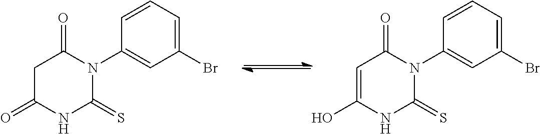 [SMILES: O=C1CC(=O)N(c2cccc(Br)c2)C(=S)N1 ; O=c1cc(O)[nH]c(=S)n1-c1cccc(Br)c1]

From 1-(3-bromophenyl) urea (200 mg, 0.93 mmoles), Diethyl malonate (353 μL), sodium methoxide (25% solution in MeOH) (531 μL). Light pale white powder; Yield: 169.7 mg, 65%. Product not very pure, however engaged in next step.

¹H NMR (500 MHZ, DMSO-d₆) δ ppm: 7.66-7.56 (m, 1H), 7.53-7.39 (m, 2H), 7.26 (d, J=7.8 Hz, 1H), 4.13 (s, 1H). ¹³C NMR (126 MHZ, DMSO-d₆) δ ppm: 168.56 (2C), 167.45, 158.34, 151.93, 132.41, 131.09, 128.96, 121.36, 66.49. HRMS (ESI+) m/z: calculated for C₁₀H₈BrN₂O₃⁺ (M+H)⁺: 282.9640; found: 282.9713.

### 1-phenyl-2-thioxodihydropyrimidine-4,6(1H,5H)-dione 2k

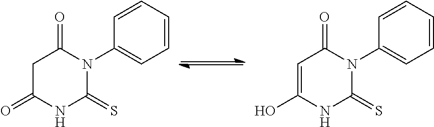 [SMILES: O=C1CC(=O)N(c2ccccc2)C(=S)N1 ; O=c1cc(O)[nH]c(=S)n1-c1ccccc1]

From 1-phenylthiourea (500 mg, 3.28 mmoles), Diethyl malonate (1250 μL), sodium methoxide (25% solution in MeOH) (1500 μL). Light pale white powder; Yield: 423 mg, 60%.

¹H NMR (400 MHZ, DMSO-d₆) δ ppm: 10.50 (bs, 1H), 7.39-7.31 (m, 2H), 7.29-7.21 (m, 1H), 7.07-6.92 (m, 2H), 4.22 (s, 1H) Enol form. ¹³C NMR (101 MHZ, DMSO-d₆) δ ppm: 176.71, 163.66, 162.82, 141.88, 130.06 (2C), 128.48 (2C), 127.06, 79.77. HRMS (ESI+) m/z: calculated for C₁₀H₉N₂O₂S⁺ (M+H)⁺: 221.0306; found: 221.0376.

### 3. Synthesis of Bis-phenyl-thiourea

The protocol was adapted from previously described procedure⁴. Fluoro/Bromo-3-isothiocyanatobenzene and 3-Fluoro/Bromoaniline in 1:1 ratio were mixed mechanically using pestle and mortar for about 15-20 minutes until creamish white color paste is formed. The crude was washed with DCM and a white color solid powder product is obtained.

### 1,3-bis(3-fluorophenyl)thiourea 3a

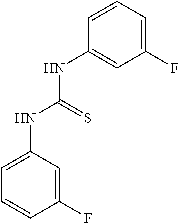 [SMILES: Fc1cccc(NC(=S)Nc2cccc(F)c2)c1]

1-fluoro-3-isothiocyanatobenzene (9 g, 7.09 mL, 58.75 mmoles), 3-fluoroaniline (5.65 mL, 1 eq); Yield: 14.38 g, 74%.

¹H NMR (400 MHZ, Acetone-d₆,) δ ppm: 9.34 (s, 2H), 7.68-7.49 (m, 2H), 7.46-7.34 (m, 2H), 7.36-7.26 (m, 2H), 6.95 (tdd, J=8.2, 2.6, 1.0 Hz, 2H). ¹³C NMR (101 MHZ, Acetone-d₆,) δ ppm: 180.24, 162.54 (d, J_{C-F}=242.9 Hz, 2C), 141.01 (d, J_{C-F}=10.6 Hz, 2C), 130.07 (d, J_{C-F}=9.5 Hz, 2C), 119.56 (d, J_{C-F}=2.9 Hz, 2C), 112.40-109.72 (m, 4C). ¹⁹F NMR 376 MHZ, Acetone-d₆,) δ ppm: −113.90. HRMS (ESI+) m/z: calculated for C₁₃H₁₁F₂N₂S⁺ (M+H)⁺: 265.0533; found: 265.0606

### 1,3-bis(3-bromophenyl)thiourea 3b

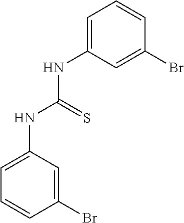 [SMILES: S=C(Nc1cccc(Br)c1)Nc1cccc(Br)c1]

1-bromo-3-isothiocyanatobenzene (100 mg, 0.47 mmoles), 3-bromoaniline (48 μL, 1 eq). Yield: 110 mg, 61%.

¹H NMR (500 MHZ, Acetone-d₆) δ ppm: 9.30 (s, 2H), 7.88 (t, J=2.0 Hz, 2H), 7.58-7.48 (m, 2H), 7.43-7.18 (m, 4H). ¹³C NMR (126 MHZ), Acetone-d₆) δ ppm: 180.53, 140.80 (2C), 130.31 (2C), 127.92 (2C), 127.01 (2C), 123.04 (2C), 121.42 (2C). LRMS (ESI+) m/z: calculated for C₁₃H₁₀Br₂N₂S⁺ (M+H)⁺: 384.9; found: 384.9

### 4. Synthesis of Diphenyl-Thiobarbituric Acid

1,3-bis(3-fluoro/bromophenyl)-2-thioxodihydropyrimidine-4,6(1H,5H)-dione was synthesized as per previously described protocol³. Briefly, to 1,3-bis(3-fluorophenyl/bromo)thiourea (1 eq) in anhydrous CHC₃, 1.2-1.3 eq of Malonic acid and 2 eq of Phosphonyl trichloride were added. The reaction mixture was put on reflux at 55° C. for about 48 hrs. The crude was then washed with H₂O and recrystallized in ethanol to obtain crystals.

### 1,3-bis(3-fluorophenyl)-2-thioxodihydropyrimidine-4,6(1H,5H)-dione 4a

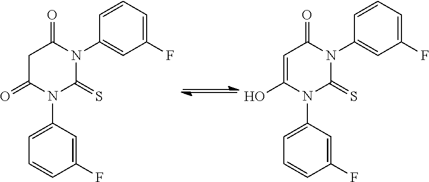 [SMILES: O=C1CC(=O)N(c2cccc(F)c2)C(=S)N1c1cccc(F)c1 ; O=c1cc(O)n(-c2cccc(F)c2)c(=S)n1-c1cccc(F)c1]

3a (1 g, 3.79 mmoles), Malonic acid (473 mg, 1.2 eq) and Phosphonyl trichloride (691 μL, 2 eq). Light yellow tinge crystals; Yield=755 mg, 60%. ¹H NMR (500 MHZ, DMSO-d₆) δ ppm: 7.39 (q, J=7.7 Hz, 2H), 7.12 (t, J=7.5 Hz, 2H), 7.05-6.91 (m, 4H), 4.48 (s, 1H) (Enol), 3.18 (s, 1H) (Keto). ¹³C NMR (126 MHZ, DMSO-d₆) δ ppm: 178.71, 162.53 (d, J_{C-F}=242.4 Hz, 2C), 162.46 (2C), 144.00 (2C), 129.97 (d, J_{C-F}=8.8 Hz, 2C), 126.46 (2C), 117.38 (d, J_{C-F}=22.4 Hz, 2C), 114.15 (d, J_{C-F}=20.7 Hz, 2C), 80.03 (Enol), 49.07 (Keto). ¹⁹F NMR (471 MHz, DMSO-d₆) δ ppm: −114.51. HRMS (ESI+) m/z: calculated for C₁₆H₁₁F₂N₂O₂S⁺(M+H)⁺: 333.0431; found: 333.0504.

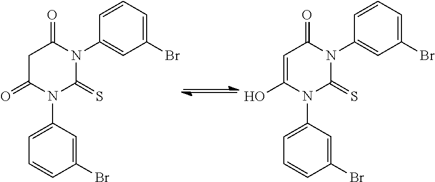 [SMILES: O=C1CC(=O)N(c2cccc(Br)c2)C(=S)N1c1cccc(Br)c1 ; O=c1cc(O)n(-c2cccc(Br)c2)c(=S)n1-c1cccc(Br)c1]

### 1,3-bis(3-bromophenyl)-2-thioxodihydropyrimidine-4,6(1H,5H)-dione 4b

3b (62 mg, 0.16 mmoles), Malonic acid (22 mg, 1.3 eq) and Phosphonyl trichloride (35 μL, 2 eq). Yellow crystals; Yield=51.5 mg, 70%.

¹H NMR (400 MHZ, Acetone-d₆) δ ppm: 7.45-7.39 (m, 2H), 7.35 (dt, J=8.9, 1.9 Hz, 2H), 7.29 (t, J=7.9 Hz, 2H), 7.17 (t, J=7.2 Hz, 2H), 4.61 (s, 1H). ¹³C NMR (101 MHZ, Acetone-d₆) δ ppm: 179.24, 162.79, 144.33 (2C), 132.98 (2C), 129.58 (2C), 129.35 (2C), 129.13 (2C), 120.58 (2C), 79.78. HRMS (ESI+) m/z: calculated for C₁₆H₁₁Br₂N₂O₂S⁺ (M+H)⁺: 452.8830; found: 454.8882.

### 5. Synthesis of Analogues with Alkyne Group by Knoevenagel Condensation

Step 1: Two methods were used:
Method A: The thiobarbituric acid derivatives (1 eq) and the 5-((trimethylsilyl) ethynyl) furan-2-carbaldehyde (1 eq) was dissolved in dry MeOH. To the reaction mixture was added catalytic amount of pyridine (1-2 drops) and the reaction was stirred at 60° C. for 2 hours. Then, the solvent and pyridine were removed under reduced pressure to obtain the crude product.
Method B: The thiobarbituric acid derivatives (1 eq) and the 5-((trimethylsilyl) ethynyl) furan-2-carbaldehyde (1 eq) was dissolved in distilled water and refluxed for 1 hour. Then, the water was removed under reduced pressure to obtain the crude product.

Step 2: The crude product from step 1 was dissolved in dry MeOH (2 ml) and K₂CO₃ (2 eq) was added. The reaction was stirred for 2 hours at r.t. and the solvent was removed under reduced pressure. The crude product was purified by preparative HPLC using acetonitrile and water and the solvents were removed under reduced pressure at 30-33° C. as freeze drying leads to degradation of the product. In some analogues, flash chromatography on silica gel using a CombiFlash system were used for the purification.

### (1-(3-bromophenyl)-5-((5-ethynylfuran-2-yl)methylene)-2-thioxodihydropyrimidine-4,6(1H,5H)-dione) 5a

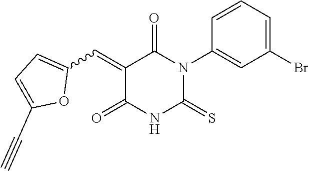 [SMILES: C#Cc1ccc(C=C2C(=O)NC(=S)N(c3cccc(Br)c3)C2=O)o1]

Using Method B of Step 1; 2g (46 mg, 0.15 mmoles) and 1 (41 mg, 1.02 eq) were dissolved in distilled water (2 mL), Brown powder, Yield=4.9 mg, 7.9% (E/Z mixture; 50:50).

¹H NMR (500 MHZ, THF-d₈) δ ppm: 12.04 (bs, 1H)/11.98 (bs, 1H) (E/Z), 8.74 (d, J=3.9 Hz, 1H)/8.55 (d, J=3.9 Hz, 1H) (E/Z), 8.25 (s, 1H)/8.18 (s, 1H) (E/Z), 7.59 (t, J=7.1 Hz, 2H), 7.48 (dt, J=13.7, 2.0 Hz, 2H), 7.39 (m, 2H), 7.25 (m, 2H), 7.06 (d, J=3.9 Hz, 1H)/7.01 (d, J=3.9 Hz, 1H) (E/Z), 4.48 (s, 1H)/4.47 (s, 1H) (E/Z).

¹³C NMR (126 MHZ, THF-d₈) δ ppm: 179.37, 161.36/159.79/159.71/158.60 (E/Z, 2C), 151.45/151.30 (E/Z), 142.43, 140.70/140.45 (E/Z), 137.49/137.16 (E/Z), 132.39/132.25 (E/Z), 131.23, 130.07, 128.39/128.21 (E/Z), 127.59/127.54 (E/Z), 121.52/121.50 (E/Z), 120.13/120.11 (E/Z), 114.49/114.29 (E/Z), 87.54, 72.91.

HRMS (ESI+) m/z: calculated for C₁₇H₁₀BrN₂O₃S⁺ (M+H)⁺: 402.2340; found: 402.9570

### 5-((5-ethynylfuran-2-yl)methylene)-1-(3-fluorophenyl)-2-thioxodihydropyrimidine-4,6(1H,5H)-dione 5b

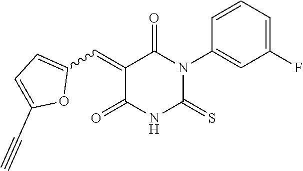 [SMILES: C#Cc1ccc(C=C2C(=O)NC(=S)N(c3cccc(F)c3)C2=O)o1]

Using Method A of Step 1: 2a (100 mg, 0.42 mmoles) and 1 (81 mg, 1.01 eq) was dissolved in anhydrous MeOH (2 mL). Brown powder, Yield: 25.5 mg, 17.8% (E/Z mixture; 50:50).

¹H NMR (500 MHZ, THF-d₈) δ ppm: 12.04 (bs, 1H)/11.98 (bs, 1H) (E/Z), 8.74 (d, J=3.9 Hz, 1H)/8.54 (d, J=4.0 Hz, 1H) (E/Z), 8.25 (s, 1H)/8.19 (s, 1H) (E/Z), 7.50-7.44 (m, 2H), 7.22-7.16 (m, 2H), 7.12-7.00 (m, 6H), 4.48 (s, 1H)/4.47 (s, 1H) (E/Z).

¹³C NMR (126 MHZ, THF-d₈) δ ppm: 179.34/179.25 (E/Z), 162.87 (d, J_{C-F}=245.1 Hz), 161.34/159.77 (E/Z), 159.73/158.62 (E/Z), 151.45/151.31 (E/Z), 142.41, 140.78 (d, J_{C-F}=10.4 Hz)/140.53 (d, J_{C-F}=10.3 Hz) (E/Z), 137.48/137.12 (E/Z), 129.82 (d, J_{C-F}=8.7 Hz), 127.52, 125.32 (d, J_{C-F}=21.1 Hz), 120.12, 116.73 (d, J_{C-F}=23.7 Hz)/116.58 (d, J_{C-F}=23.4 Hz) (E/Z), 115.13/114.96 (E/Z), 114.43 (d, J=21.8 Hz), 87.54, 72.92.

¹⁹F NMR (471 MHZ, THF-d₈) δ ppm: −114.11.

HRMS (ESI+) m/z: calculated for C₁₇H₁₀FN₂O₃S⁺ (M+H)⁺: 341.0318; found: 341.0391.

### 5-((5-ethynylfuran-2-yl)methylene)-1-(2-fluorophenyl)-2-thioxodihydropyrimidine-4,6(1H,5H)-dione 5c

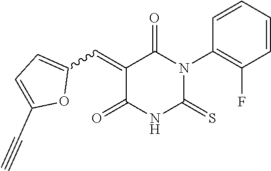 [SMILES: C#Cc1ccc(C=C2C(=O)NC(=S)N(c3ccccc3F)C2=O)o1]

Using Method A of Step 1: 2c (50 mg, 0.21 mmoles) and 1 (41 mg, 1.02 eq) was dissolved in anhydrous MeOH (2 mL). Brown powder, Yield: 5.4 mg, 7.6% (E/Z mixture; 50:50).

¹H NMR (500 MHZ, THF-d₈) δ ppm: 12.13 (bs, 1H)/12.06 (bs, 1H) (E/Z), 8.76 (d, J=3.9 Hz, 1H)/8.55 (d, J=3.9 Hz, 1H) (E/Z), 8.28 (s, 1H)/8.20 (s, 1H) (E/Z), 7.46 (t, J=6.9 Hz, 2H), 7.37-7.30 (m, 2H), 7.29-7.22 (m, 4H), 7.04 (dd, J=26.9 (E/Z), 3.9 Hz, 2H), 4.48 (s, 2H).

¹³C NMR (126 MHZ, THE-dg) δ ppm: 178.73, 160.89/159.27 (E/Z),159.6, 158.23 (d, J_{C-F}=233.0 Hz),151.46/151.31 (E/Z), 142.55, 137.90/137.60 (E/Z), 131.06 (d, J_{C-F}=20.7 Hz), 130.37 (d, J_{C-F}=6.7 Hz), 127.75, 124.21, 120.15, 115.79 (d, J_{C-F}=19.8 Hz), 114.46, 114.01/113.80 (E/Z), 87.59, 72.91.

¹⁹F NMR (471 MHZ, THF-d₈) δ ppm: −122.65/−122.77 (E/Z).

HRMS (ESI+) m/z: calculated for C₁₇H₁₀FN₂O₃S⁺ (M+H)⁺: 341.0318; found: 341.0391.

### 5-((5-ethynylfuran-2-yl)methylene)-1-(4-fluorophenyl)-2-thioxodihydropyrimidine-4,6(1H,5H)-dione 5d

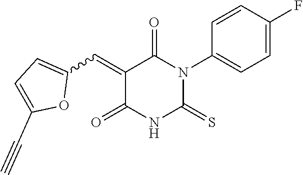 [SMILES: C#Cc1ccc(C=C2C(=O)NC(=S)N(c3ccc(F)cc3)C2=O)o1]

Using Method A of Step 1: 2b (50 mg, 0.21 mmoles) and 1 (41 mg, 1.01 eq) was dissolved in anhydrous MeOH (2 mL). Brown powder, Yield: 6.7 mg, 9.3% (E/Z mixture; 50:50).

¹H NMR (500 MHZ, THF-d₈) δ ppm: 12.02 (bs, 1H)/11.96 (bs, 1H) (E/Z), 8.73 (d, J=3.9 Hz, 1H)/8.54 (d, J=3.9 Hz, 1H) (E/Z), 8.24 (s, 1H)/8.18 (s, 1H) (E/Z), 7.29-7.24 (m, 4H), 7.22-7.18 (m, 4H), 7.06 (d, J=3.9 Hz, 1H)/7.01 (d, J=3.9 Hz, 1H) (E/Z), 4.47 (s, 2H).

¹³C NMR (126 MHZ, THF-d₈) δ ppm: 179.70/179.60 (E/Z), 162.37 (d, J_{C-F}=245.7 Hz), 161.52/158.61 (E/Z), 159.96/159.71 (E/Z), 151.47/151.33 (E/Z), 142.35, 137.47/137.05 (E/Z), 135.47/135.19 (E/Z), 131.10 (d, J_{C-F}=8.9 Hz), 130.93 (d, J_{C-F}=8.7 Hz), 127.42, 120.09, 115.39 (d, J_{C-F}=23.1 Hz, 2C), 114.61/114.45 (E/Z), 87.49, 72.92. ¹⁹F NMR (471 MHZ, THF-d₈) δ ppm: −115.05/−115.06 (E/Z). HRMS (ESI+) m/z: calculated for C₁₇H₁₀FN₂O₃S⁺ (M+H)⁺: 341.0318; found: 341.0391.

### 1-(2,5-difluorophenyl)-5-((5-ethynylfuran-2-yl)methylene)-2-thioxodihydropyrimidine-4,6(1H,5H)-dione 5e

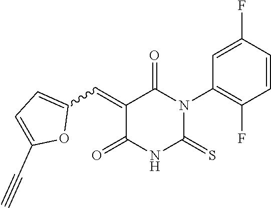 [SMILES: C#Cc1ccc(C=C2C(=O)NC(=S)N(c3cc(F)ccc3F)C2=O)o1]

Using Method A of Step 1: 2f (100 mg, 0.39 mmoles) and 1 (75 mg, 1.01 eq) was dissolved in anhydrous MeOH (3 mL). Purified by Flash chromatography in (cHex/EtOAc). Brown powder, Yield: 14.3 mg, 10.2%. Traces of grease in sample due to cHex (E/Z mixture; 50:50).

¹H NMR (500 MHZ, THF-d₈) δ ppm: 12.19 (bs, 1H)/12.13 (bs, 1H) (E/Z), 8.76 (d, J=3.9 Hz, 1H)/8.56 (d, J=3.9 Hz, 1H) (E/Z), 8.29 (s, 1H)/8.21 (s, 1H) (E/Z), 7.33-7.23 (m, 4H), 7.22-7.14 (m, 2H), 7.08 (d, J=3.9 Hz, 1H)/7.03 (d, J=3.9 Hz, 1H) (E/Z), 4.49 (s, 2H).

¹³C NMR (126 MHZ, THF-d₈) δ ppm: 178.58/178.45 (E/Z), 160.83/159.64 (E/Z), 158.41 (d, J_{C-F}=242.6 Hz), 159.17/158.54 (E/Z), 154.85 (d, J=248.1 Hz)/154.72 (d, J=245.7 Hz) (E/Z), 151.41/151.25 (E/Z), 142.76/142.71 (E/Z), 138.05/137.83 (E/Z), 128.03/127.96 (E/Z), 127.57-127.00 (m), 120.22, 118.02 (d, J=17.8 Hz)/117.82 (d, J=17.7 Hz) (E/Z), 117.09-116.60 (m, 2C), 113.76/113.54 (E/Z), 87.76/87.73 (E/Z), 72.88.

¹⁹F NMR (471 MHZ, THF-d₈) δ ppm: −119.29 (d, J=2.9 Hz)/−119.32 (d, J=2.7 Hz) (E/Z), −127.66 (d, J_{F-F}=16.2 Hz)/−127.80 (d, J_{F-F}=16.2 Hz) (E/Z).

HRMS (ESI+) m/z: calculated for C₁₇H₉F₂N₂O₃S⁺ (M+H)⁺: 358.0224; found: 359.0296.

### 5-((5-ethynylfuran-2-yl)methylene)-1-(3-methoxyphenyl)-2-thioxodihydropyrimidine-4,6(1H,5H)-dione 5f

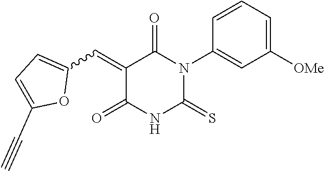 [SMILES: C#Cc1ccc(C=C2C(=O)NC(=S)N(c3cccc(OC)c3)C2=O)o1]

Using Method A of Step 1: 2d (43 mg, 0.17 mmoles) and 1 (33 mg, 1 eq) was dissolved in anhydrous MeOH (2 mL). Brown powder, Yield: 5.4 mg, 8.9% (E/Z mixture; 50:50).

¹H NMR (500 MHZ, THF-d₈) δ ppm: 11.96 (bs, 1H)/11.90 (bs, 1H) (E/Z), 8.73 (d, J=3.9 Hz, 1H)/8.55 (d, J=3.9 Hz, 1H) (E/Z), 8.24 (s, 1H)/8.17 (s, 1H) (E/Z), 7.33 (td, J=8.0, 5.3 Hz, 2H), 7.05 (d, J=3.9 Hz, 1H)/7.00 (d, J=4.0 Hz, 1H) (E/Z), 6.99-6.94 (m, 2H), 6.85-6.78 (m, 4H), 4.46 (s, 2H), 3.80 (s, 6H).

¹³C NMR (126 MHZ, THF-d₈) δ ppm: 179.31, 161.24/158.62 (E/Z), 160.38, 159.72, 151.51/151.37 (E/Z), 142.22, 140.35/140.06 (E/Z), 137.33/136.90 (E/Z), 129.02, 127.31/127.24 (E/Z), 121.25/121.09 (E/Z), 120.04, 114.85/114.73 (E/Z),114.56, 113.77, 87.38, 72.95, 54.53.

HRMS (ESI+) m/z: calculated for C₁₈H₁₃N₂O₄S⁺ (M+H)⁺: 353.0518; found: 353.0591

### 5-((5-ethynylfuran-2-yl)methylene)-1-(2-methoxyphenyl)-2-thioxodihydropyrimidine-4,6(1H,5H)-dione 5g

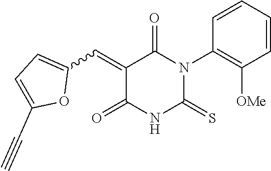 [SMILES: C#Cc1ccc(C=C2C(=O)NC(=S)N(c3ccccc3OC)C2=O)o1]

Using Method A of Step 1: 2e (30 mg, 0.12 mmoles) and 1 (23 mg, 1 eq) was dissolved in anhydrous MeOH (2 mL). Brown powder, Yield: 5.9 mg, 13.9% (E/Z mixture; 50:50).

¹H NMR (500 MHZ, THF-d₈) δ ppm: 11.97 (s, 1H)/11.90 (s, 1H) (E/Z), 8.74 (d, J=4.0 Hz, 1H)/8.54 (d, J=3.9 Hz, 1H) (E/Z), 8.25 (s, 1H)/8.17 (s, 1H) (E/Z), 7.38 (q, J=7.1, 6.5 Hz, 2H), 7.20-7.13 (m, 2H), 7.12-7.04 (m, 2H), 7.03-6.96 (m, 3H), 4.46 (s, 2H), 3.78 (s, 6H).

¹³C NMR (126 MHZ, THF-d₈) δ ppm: 179.08/179.00 (E/Z), 160.73/159.71 (E/Z), 159.22/158.62 (E/Z), 155.27/155.12 (E/Z), 151.54/151.40 (E/Z), 142.29/142.24 (E/Z), 137.69/137.27 (E/Z), 130.13/129.99 (E/Z), 129.61, 127.88/127.58 (E/Z), 127.42/127.33 (E/Z), 120.13/120.02 (E/Z), 114.32/114.15 (E/Z), 111.67, 87.38, 72.95, 54.97.

HRMS (ESI+) m/z: calculated for C₁₈H₁₃N₂O₄S⁺ (M+H)⁺: 353.0518; found: 353.0591.

### 5-((5-ethynylfuran-2-yl)methylene)-2-thioxo-1-(3-(trifluoromethyl)phenyl)dihydropyrimidine-4,6(1H,5H)-dione 5h

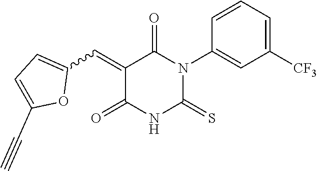 [SMILES: C#Cc1ccc(C=C2C(=O)NC(=S)N(c3cccc(C(F)(F)F)c3)C2=O)o1]

Using Method A of Step 1: 2h (30 mg, 0.11 mmoles) and 1 (23 mg, 1 eq) was dissolved in anhydrous MeOH (2 mL). Brown powder, Yield: 5.9 mg, 13.9% (E/Z mixture; 50:50).

¹H NMR (500 MHz Acetone-d₆) δ ppm: 11.80 (bs, 1H)/11.75 (bs, 1H) (E/Z), 8.72 (d, J=3.9 Hz, 1H)/8.50 (d, J=4.0 Hz, 1H) (E/Z), 8.21 (s, 1H)/8.15 (s, 1H) (E/Z), 8.31-8.10 (m, 2H), 7.88-7.77 (m, 6H), 7.72 (ddt, J=13.4, 8.0, 1.6 Hz, 2H), 7.21 (dd, J=4.0, 0.8 Hz, 1H)/7.15 (dd, J=4.0, 0.8 Hz, 1H) (E/Z), 4.67 (s, 1H)/4.67 (s, 1H) (E/Z).

¹³C NMR (126 MHZ), Acetone-d₆) δ ppm: 179.63/179.52 (E/Z), 161.95/158.69 (E/Z), 159.98/160.16 (E/Z), 151.27/151.13 (E/Z), 142.35, 140.35/140.11 (E/Z), 137.90/137.50 (E/Z), 133.66/133.49 (E/Z), 131.08-130.51 (m), 130.21/130.16 (E/Z), 128.11/128.02 (E/Z), 126.50/126.36 (E/Z), 125.34, 124.12 (d, J_{C-F}=271.5 Hz), 120.76, 114.67/114.55 (E/Z), 88.21, 72.85.

¹⁹F NMR (471 MHz, Acetone-d₆) δ ppm: −63.02/−63.04 (E/Z).

HRMS (ESI+) m/z: calculated for C₁₈H₁₀F₃N₂O₃S⁺ (M+H)⁺: 391.0286; found: 391.0359.

### 1-(3,5-bis(trifluoromethyl)phenyl)-5-((5-ethynylfuran-2-yl)methylene)-2-thioxodihydropyrimidine-4,6(1H,5H)-dione 5i

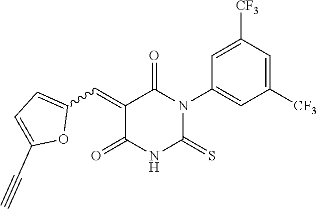 [SMILES: C#Cc1ccc(C=C2C(=O)NC(=S)N(c3cc(C(F)(F)F)cc(C(F)(F)F)c3)C2=O)o1]

Using Method A of Step 1: 2i (41 mg, 0.12 mmoles and 1 (22 mg, 1 eq) was dissolved in anhydrous MeOH (2 mL). Brown powder, Yield: 5.2 mg, 9.8% (E/Z mixture; 50:50).

¹H NMR (500 MHZ, THF-d₈) δ ppm: 12.24 (bs, 1H)/12.18 (bs, 1H) (E/Z), 8.77 (d, J=4.0 Hz, 1H)/8.55 (d, J=3.9 Hz, 1H) (E/Z), 8.31 (s, 1H)/8.22 (s, 1H) (E/Z), 8.15 (d, J=7.1 Hz, 2H), 8.00 (s, 2H), 7.97 (s, 2H), 7.10 (d, J=3.9 Hz, 1H)/7.04 (d, J=3.9 Hz, 1H) (E/Z), 4.52 (s, 1H)/4.51 (s, 1H) (E/Z).

¹³C NMR (126 MHZ, THF-d₈) δ ppm: 179.36/179.23 (E/Z), 161.57/158.57 (E/Z), 159.90/159.69 (E/Z), 151.33/151.17 (E/Z), 142.86, 140.97, 137.91137.73 (E/Z), 132.02 (d, J_{C-F}=34.2 Hz, 2C), 130.65/130.49 (E/Z), 128.19, 123.26 (d, J_{C-F}=272.4 Hz, 2C), 122.28, 120.32/120.28 (E/Z), 113.97/113.75 (E/Z), 87.88, 72.84.

¹⁹F NMR (471 MHZ, THF-d₈) δ ppm: −63.57, −63.59.

HRMS (ESI+) m/z: calculated for C₁₉H₉F₆N₂O₃S⁺ (M+H)⁺: 459.0160; found: 459.0233.

### 1,3-bis(3-bromophenyl)-5-((5-ethynylfuran-2-yl)methylene)-2-thioxodihydropyrimidine-4,6(1H,5H)-dione 5j

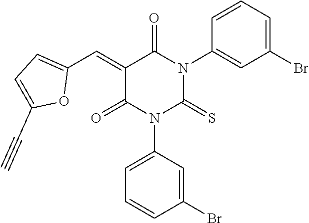 [SMILES: C#Cc1ccc(C=C2C(=O)N(c3cccc(Br)c3)C(=S)N(c3cccc(Br)c3)C2=O)o1]

Using Method B of Step 1: 4b (30 mg, 0.07 mmoles) and 1 (12 mg, 1 eq) was dissolved in distilled water (2 mL). Brown powder, Yield: 4.8 mg, 13.06%.

¹H NMR (500 MHZ, THF-d₈) δ ppm: 8.62 (d, J=4.0 Hz, 1H), 8.31 (s, 1H), 7.65-7.56 (m, 2H), 7.50 (dt, J=13.4, 2.0 Hz, 2H), 7.45-7.38 (m, 2H), 7.32-7.22 (m, 2H), 7.05 (d, J=3.9 Hz, 1H), 4.50 (s, 1H).

¹³C NMR (126 MHZ, THF-d₈) δ ppm: 180.22, 160.51, 159.00, 151.36, 142.86, 141.46/141.18 (2C), 138.42, 132.17/132.10 (2C), 131.20 (2C), 130.26 (2C), 128.17 (2C), 128.05, 121.69, 120.29, 114.22, 87.87, 72.86.

HRMS (ESI+) m/z: calculated for C₂₃H₁₃Br₂N₂O₃S⁺ (M+H)⁺: 557.2280; found: 556.8988.

### 5-((5-ethynylfuran-2-yl)methylene)-1,3-bis(3-fluorophenyl)-2-thioxodihydropyrimidine-4,6(1H,5H)-dione 5k

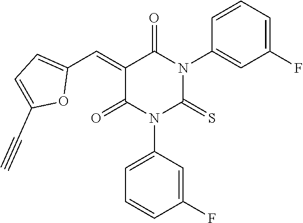 [SMILES: C#Cc1ccc(C=C2C(=O)N(c3cccc(F)c3)C(=S)N(c3cccc(F)c3)C2=O)o1]

Using Method B of Step 1: 4a (296 mg, 0.89 mmoles) and 1 (171 mg, 1 eq) was dissolved in distilled water (5 mL). Orange Brown powder, Yield: 54.3 mg, 14.1%.

¹H NMR (500 MHZ, THF-d₈) δ ppm: 8.61 (d, J=4.0 Hz, 1H), 8.31 (s, 1H), 7.54-7.41 (m, 2H), 7.23-7.15 (m, 2H), 7.15-7.06 (m, 4H), 7.05 (d, J=3.9 Hz, 1H), 4.50 (s, 1H).

¹³C NMR (126 MHZ, THF-d₈) δ ppm: 180.14, 163.00 (d, J_{C-F}=245.3 Hz, 2C), 160.49, 159.00, 151.38, 142.77, 141.57 (d, J_{C-F}==10.6 Hz), 141.29 (d, J_{C-F}==10.4 Hz), 138.31, 130.00 (d, J_{C-F}=8.8 Hz, 2C), 128.00, 125.18 (d, J_{C-F}=21.9 Hz, 2C), 120.26, 116.60 (d, J_{C-F}==18.8 Hz), 116.42 (d, J_{C-F}=19.0 Hz), 115.00 (d, J_{C-F}=21.1 Hz, 2C), 114.37, 87.81, 72.87.

¹⁹F NMR (471 MHZ, THF-d₈) δ ppm: −113.91, −113.94.

HRMS (ESI+) m/z: calculated for C₂₃H₁₃F₂N₂O₃S⁺ (M+H)⁺: 435.0537; found: 435.0609.

### 1-(3-bromophenyl)-5-((5-ethynylfuran-2-yl)methylene)pyrimidine-2,4,6(1H,3H,5H)-trione 5l

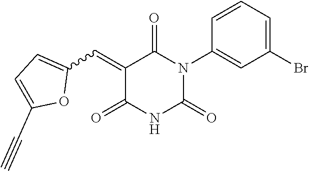 [SMILES: C#Cc1ccc(C=C2C(=O)NC(=O)N(c3cccc(Br)c3)C2=O)o1]

Using Method A of Step 1: 4j (67.8 mg, 0.24 mmoles) and 1 (46.5 mg, 1 eq) was dissolved in anhydrous MeOH (2 mL). Brown powder powder, Yield: 8.5 mg, 9.2% (E/Z mixture; 50:50).

¹H NMR (500 MHZ, Acetone-d₆) δ ppm: 10.55 (bs, 1H)/10.48 (bs, 1H) (E/Z), 8.63 (d, J=3.9 Hz, 1H)/8.46 (d, J=3.8 Hz, 1H) (E/Z), 8.20 (s, 1H)/8.16 (s, 1H) (E/Z), 7.76-7.63 (m, 4H), 7.54-7.42 (m, 4H), 7.14 (dd, J=4.0, 0.8 Hz, 1H)/7.09 (dd, J=4.0, 0.8 Hz, 1H) (E/Z), 4.52 (s, 1H)/4.52 (s, 1H) (E/Z).

¹³C NMR (126 MHZ, Acetone-d₆) δ ppm: 161.68, 161.41, 160.81, 151.11/151.00 (E/Z), 149.72/149.63 (E/Z), 141.77, 137.32/137.00 (E/Z), 132.41/132.25 (E/Z), 131.46, 130.51, 128.57/128.40 (E/Z), 127.10/126.96 (E/Z), 121.31/121.25 (E/Z), 120.29, 114.56/114.47 (E/Z), 87.43, 72.84.

HRMS (ESI+) m/z: calculated for C₁₇H₁₀BrN₂O₄+ (M+H)⁺: 384.9746; found: 384.9818.

### 6. Synthesis of Analogues by Knoevenagel Condensation in Acidic Condition.⁷

1 equivalent of thiourea derivatives was dissolved in 2 mL of acetic acid, the solution became yellow in most cases. Furan derivative (1 eq) was added and the solution mixture was stirred at rt for 15 minutes to 1 hour. Completion of the reaction was followed by TLC in 2 solvents systems (Hex/EA and DCM/MeOH to make sure the starting material was converted) and by UPLC-MS. When the reaction was completed, the solvent was evaporated in vacuo, and the crude solid was mixed with 10 ml of a mixture of cHex/EtOAc (4:6) and sonicated. The resultant solution was filtered with the same cHex/EtOAc mixture as before. The liquid was then evaporated to obtain a pure product. Further purifications are carried out for impure product and purification methods are written against the analogues.

### 1-(3-fluorophenyl)-5-((5-methylfuran-2-yl)methylene)-2-thioxodihydropyrimidine-4,6(1H,5H)-dione 6a

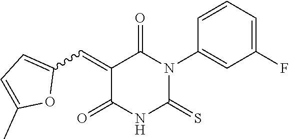 [SMILES: Cc1ccc(C=C2C(=O)NC(=S)N(c3cccc(F)c3)C2=O)o1]

50 mg (0.21 mmoles) of 2a and 5-methylfuran-2-carbaldehyde (23.5 mg, 1 eq) were dissolved in 2 mL of acetic acid. Reaction time=15 minutes. Yellow-orange powder, Yield: 9.2 mg, 13.2% (E/Z mixture; 50:50).

¹H NMR (500 MHZ, Acetone-d₆) δ ppm: 11.58 (bs, 1H)/11.52 (bs, 1H) (E/Z), 8.77 (d, J=3.8 Hz, 1H)/8.57 (d, J=3.8 Hz, 1H) (E/Z), 8.25 (s, 1H), 8.18 (s, 1H) (E/Z), 7.67-7.48 (m, 2H), 7.39-7.12 (m, 6H), 6.69 (dt, J=3.8, 0.8 Hz, 1H)/6.64 (dt, J=3.8, 0.8 Hz, 1H) (E/Z), 2.53 (s, 6H).

¹³C NMR (126 MHZ, Acetone-d₆) δ ppm: 179.42/179.33 (E/Z), 164.44/163.74 (E/Z), 162.74 (d, J_{C-F}=243.9 Hz), 161.80/160.22 (E/Z), 160.40/158.92 (E/Z), 150.35/150.20 (E/Z), 141.08 (d, J_{C-F}=10.5 Hz), 138.91/138.46 (E/Z), 130.33 (d, J_{C-F}=8.9 Hz), 125.78/125.62 (E/Z), 116.78 (d, J_{C-F}=23.5 Hz), 115.26 (d, J_{C-F}=21.1 Hz), 113.59, 110.83/110.73 (E/Z), 13.69.

HRMS (ESI+) m/z: calculated for C₁₆H₁₂FN₂O₃S⁺ (M+H)⁺: 331.0474; found: 331.0547

### 5-((5-chlorofuran-2-yl)methylene)-1-(3-fluorophenyl)-2-thioxodihydropyrimidine-4,6(1H,5H)-dione 6b

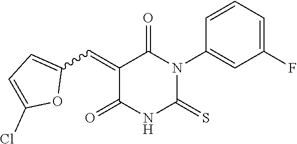 [SMILES: O=C1NC(=S)N(c2cccc(F)c2)C(=O)C1=Cc1ccc(Cl)o1]

50 mg (0.21 mmoles) of 2a and 5-methylfuran-2-carbaldehyde (27.5 mg, 1 eq) were dissolved in 2 ml of acetic acid. Reaction time=25 minutes. Yellow-orange powder, Yield: 8.2 mg, 11.1% (E/Z mixture; 50:50).

¹H NMR (Acetone-d₆, 500 MHz) δ ppm: 11.71 (bs, 1H)//11.66 (bs, 1H) (E/Z), 8.75 (d, J=3.9 Hz, 1H)/8.56 (d, J=4.0 Hz, 1H) (E/Z), 8.18 (s, 1H)/8.12 (s, 1H) (E/Z), 7.64-7.53 (m, 2H), 7.37-7.16 (m, 6H), 6.94 (dd, J=3.9, 0.7 Hz, 1H)/6.88 (dd, J=4.0, 0.7 Hz, 1H) (E/Z).

¹³C NMR (126 MHz, Acetone-d₆) δ ppm: 179.46/179.38 (E/Z), 162.72 (d, J_{C-F}=246.2 Hz), 161.75/159.95 (E/Z), 160.10/158.77 (E/Z), 151.05/150.93 (E/Z), 145.22, 141.00 (d, J_{C-F}=10.0 Hz)/140.73 (d, J_{C-F}=10.4 Hz) (E/Z), 137.72/137.29 (E/Z), 130.45 (d, J_{C-F}=5.8 Hz)/130.38 (d, J_{C-F}=5.6 Hz) (E/Z), 130.23/130.14 (E/Z), 125.70/125.54 (E/Z), 116.80 (d, J_{C-F}=19.8 Hz)/116.61 (d, J_{C-F}=19.9 Hz) (E/Z), 115.40 (d, J_{C-F}=21.0 Hz), 113.55/113.45 (E/Z), 113.00.

HRMS (ESI+) m/z: calculated for C₁₅H₉ClFN₂O₃S⁺ (M+H)⁺: 350.9928; found: 351.0001.

### 5-((5-chlorofuran-2-yl)methylene)-1-(3-bromophenyl)-2-thioxodihydropyrimidine-4,6(1H,5H)-dione 6c

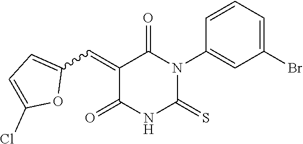 [SMILES: O=C1NC(=S)N(c2cccc(Br)c2)C(=O)C1=Cc1ccc(Cl)o1]

60 mg (0.20 mmoles) of 2g and 5-chlorofuran-2-carbaldehyde (20 mg, 1 eq) were dissolved in 2 mL of acetic acid. Reaction time=45 minutes. Yellow powder, Yield: 52.1 mg, 63.1% (E/Z mixture; 50:50). 1H NMR (500 MHZ, THF-da) δ ppm: 12.04 (bs, 1H)/11.98 (bs, 1H) (E/Z), 8.79 (d, J=3.9 Hz, 1H)/8.60 (d, J=3.9 Hz, 1H) (E/Z), 8.24 (s, 1H)/8.17 (s, 1H) (E/Z), 7.59 (t, J=6.2, 2H), 7.48 (d, J=14.0 Hz, 2H), 7.44-7.35 (m, 2H), 7.29-7.19 (m, 2H), 6.81 (d, J=3.9 Hz, 1H)/6.76 (d, J=3.9 Hz, 1H) (E/Z).

¹³C NMR (126 MHZ, THF-dg) δ ppm: 179.37/179.27 (E/Z), 161.34/159.90 (E/Z), 159.69/158.72 (E/Z), 151.30/151.18 (E/Z), 145.03/144.98 (E/Z), 140.69/140.43 (E/Z), 137.35/137.01 (E/Z), 132.39/132.25 (E/Z), 131.24, 130.08, 129.75/129.68 (E/Z), 128.39/128.21 (E/Z), 121.53, 113.51/113.33 (E/Z), 112.48.

HRMS (ESI+) m/z: calculated for C₁₅H₉BrClN₂O₃S⁺ (M+H)⁺: 412.6540; found: 412.9180.

### 1-(3-fluorophenyl)-5-((5-nitrofuran-2-yl)methylene)-2-thioxodihydropyrimidine-4,6(1H,5H)-dione 6e

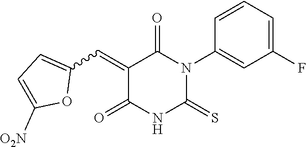 [SMILES: O=C1NC(=S)N(c2cccc(F)c2)C(=O)C1=Cc1ccc([N+](=O)[O-])o1]

50 mg (0.21 mmoles) of 2a and 5-nitrofuran-2-carbaldehyde (23 mg, 1 eq) were dissolved in 2 mL of acetic acid. Reaction time=25 minutes. Purified by flash chromatography on silica using EtOAc-cHex. Yellow powder, Yield: 4 mg, 5.2% (E/Z mixture; 50:50).

¹H NMR (500 MHZ, THF-d₈) δ ppm: 12.23 (bs, 1H)/12.17 (bs, 1H) (E/Z), 8.70 (d, J=4.1 Hz, 1H)/8.52 (d, J=4.1 Hz, 1H) (E/Z), 8.27 (s, 1H)/8.21 (s, 1H) (E/Z), 7.67 (d, J=4.1 Hz, 1H)/7.64 (d, J=4.1 Hz, 1H) (E/Z), 7.56-7.40 (m, 2H), 7.31-7.17 (m, 2H), 7.14-7.00 (m, 4H).

¹³C NMR (126 MHZ, THF-d₈) δ ppm: 179.32/179.23 (E/Z), 162.87 (d, J_{C-F}=242.8 Hz), 160.69/159.63 (E/Z), 159.03/158.38 (E/Z), 154.03, 150.89/150.76 (E/Z), 140.34 (d, J_{C-F}=21.9 Hz), 136.41 (d, J_{C-F}=41.0 Hz), 130.02/129.95 (E/Z), 126.30, 125.21 (d, J_{C-F}=20.6 Hz), 119.78/119.58 (E/Z), 116.63 (d, J_{C-F}=17.3 Hz)/116.44 (d, J_{C-F}=16.8 Hz) (E/Z), 115.28 (d, J_{C-F}=21.1 Hz), 112.74. ¹⁹F NMR (471 MHZ, THF-da) δ ppm: −113.85/−113.88 (E/Z). HRMS (ESI+) m/z: calculated for C₁₅H₉FN₃O₅S⁺ (M+H)⁺: 362.0169; found: 362.0241.

### 5-((5-bromofuran-2-yl)methylene)-1-(3-fluorophenyl)-2-thioxodihydropyrimidine-4,6(1H,5H)-dione 6i

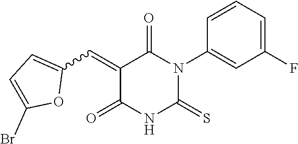 [SMILES: O=C1NC(=S)N(c2cccc(F)c2)C(=O)C1=Cc1ccc(Br)o1]

50 mg (0.21 mmoles) of 2a and 5-bromofuran-2-carbaldehyde (37 mg, 1 eq) were dissolved in 2 mL of acetic acid. Reaction time=25 minutes. Yellow powder, Yield: 8.9 mg; 10.7% (E/Z mixture; 50:50).

¹H NMR (500 MHZ, THF-d₈) δ ppm: 12.02 (bs, 1H)/11.96 (bs, 1H) (E/Z), 8.72 (d, J=3.9 Hz, 1H)/8.53 (d, J=3.9 Hz, 1H) (E/Z), 8.27 (s, 1H)/8.20 (s, 1H) (E/Z), 7.55-7.39 (m, 2H), 7.27-7.14 (m, 2H), 7.14-7.02 (m, 4H), 6.92 (d, J=3.9 Hz, 1H)/6.87 (d, J=3.9 Hz, 1H) (E/Z).

¹³C NMR (126 MHZ, THF-dg) δ ppm: 179.35, 162.82 (d, J_{C-F}==256.0 Hz), 161.31/158.73 (E/Z), 159.87/159.70 (E/Z), 153.60/153.47 (E/Z), 140.85/140.58 (E/Z), 137.25/136.89 (E/Z), 132.80/132.85, 129.79 (d, J_{C-F}=8.8 Hz), 129.48/129.46 (E/Z), 125.32 (d, J_{C-F}=19.2 Hz), 117.39, 116.72 (d, J_{C-F}=23 Hz)/116.58 (d, J_{C-F}=23.4 Hz) (E/Z), 115.01 (d, J_{C-F}=20.6 Hz), 113.31/113.15 (E/Z).

¹⁹F NMR (471 MHZ, THF-d₈) δ ppm: −114.16/−114.16 (E/Z).

HRMS (ESI+) m/z: calculated for C₁₅H₉BrFN₂O₃S⁺ (M+H)⁺: 396.2024; found: 396.9475.

### 1,3-bis(3-fluorophenyl)-5-((5-methylfuran-2-yl)methylene)-2-thioxodihydropyrimidine-4,6(1H,5H)-dione 6j

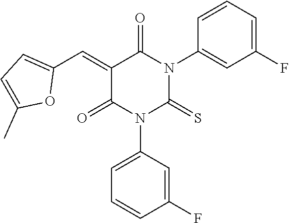 [SMILES: Cc1ccc(C=C2C(=O)N(c3cccc(F)c3)C(=S)N(c3cccc(F)c3)C2=O)o1]

100 mg (0.30 mmoles) of 4a and 5-methylfuran-2-carbaldehyde (29.6 μL, 1 eq) were dissolved in 2 mL of acetic acid. Reaction time=30 minutes. Purified by flash chromatography on silica using EtOAc-cHex. Yellow-orange powder, Yield: 28 mg, 21.8%.

¹H NMR (500 MHZ, THF-d₈) δ ppm: 8.64 (d, J=3.8 Hz, 1H), 8.33 (s, 1H), 7.53-7.32 (m, 2H), 7.28-6.95 (m, 6H), 6.52 (d, J=3.7 Hz, 1H), 2.46 (s, 3H).

¹³C NMR (126 MHZ, THF-d₈) δ ppm: 180.22, 164.21, 162.98 (d, J_{C-F}=244.8 Hz, 2C), 160.91, 159.22, 150.59, 141.80 (d, J_{C-F}=10.3 Hz), 141.54 (d, J_{C-F}=10.6 Hz), 139.40, 131.29, 129.89 (d, J_{C-F}=8.9 Hz, 2C), 125.25 (d, J_{C-F}=23.0 Hz, 2C), 116.69 (d, J_{C-F}=19.7 Hz), 116.50 (d, J_{C-F}=20.3 Hz), 114.83 (d, J_{C-F}=21.0 Hz, 2C), 113.20, 110.82, 13.38.

¹⁹F NMR (471 MHZ, THF-d₈) 5 ppm: −114.09, −114.11.

HRMS (ESI+) m/z: calculated for C₂₂H₁₅F₂N₂O₃S⁺ (M+H)⁺: 425.0693; found: 425.0766.

### 5-(furan-2-ylmethylene)-1-phenyl-2-thioxodihydropyrimidine-4,6(1H,5H)-dione 6k

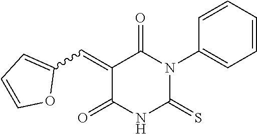 [SMILES: O=C1NC(=S)N(c2ccccc2)C(=O)C1=Cc1ccco1]

100 mg (0.45 mmoles) of 2k and furan-2-carbaldehyde (39.6 μL, 1 eq) were dissolved in 2 mL of acetic acid. Reaction time=45 minutes. Brown powder, Yield: 25 mg, 18% (E/Z mixture; 50:50).

¹H NMR (500 MHZ, THF-d₈) δ ppm: 11.93 (s, 1H)/11.87 (s, 1H) (E/Z), 8.79 (d, J=3.8 Hz, 1H)/8.59 (d, J=3.8 Hz, 1H) (E/Z), 8.36 (s, 1H)/8.28 (s, 1H) (E/Z), 8.11 (d, J=1.6 Hz, 1H)/8.09 (d, J=1.6 Hz, 1H) (E/Z), 7.50-7.35 (m, 6H), 7.30-7.18 (m, 4H), 6.86 (d, J=3.0 Hz, 1H)/6.80 (d, J=3.1 Hz, 1H) (E/Z).

¹³C NMR (126 MHZ, THF-d₈) δ ppm: 179.65/179.57 (E/Z), 161.62/158.76 (E/Z), 159.96, 151.45/151.30 (E/Z), 151.15/151.07 (E/Z), 139.58/139.31 (E/Z), 138.91/138.48 (E/Z), 129.18, 129.01, 128.51, 128.48, 127.89, 127.52/127.45 (E/Z), 114.96, 113.32/113.15 (E/Z).

HRMS (ESI+) m/z: calculated for C₁₅H₁₁N₂O₃S⁺ (M+H)⁺: 299.0412; found: 299.0485

### 1-(3-bromophenyl)-5-(furan-2-ylmethylene)pyrimidine-2,4,6(1H,3H,5H)-trione 61

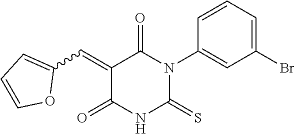 [SMILES: O=C1NC(=S)N(c2cccc(Br)c2)C(=O)C1=Cc1ccco1]

100 mg (0.35 mmoles) of 2j and furan-2-carbaldehyde (31 μL, 1.05 eq) were dissolved in 2 mL of acetic acid. Reaction time=15 minutes. Brown powder, Yield: 18 mg, 14% (E/Z mixture; 50:50).

¹H NMR (500 MHZ, DMSO-d₆) δ ppm: 11.77 (s, 1H)/11.69 (s, 1H) (E/Z), 8.55 (d, J=3.8 Hz, 1H)/8.37 (d, J=3.8 Hz, 1H) (E/Z), 8.31 (s, 1H)/8.29 (s, 1H) (E/Z8.16 (s, 1H)/8.09 (s, 1H) (E/Z), 7.72-7.56 (m, 4H), 7.52-7.43 (m, 2H), 7.42-7.32 (m, 2H), 6.96 (s, 1H)/6.89 (s, 1H) (E/Z).

¹³C NMR (126 MHZ, DMSO-d₆) δ ppm: 162.92, 162.34, 161.36/161.26 (E/Z), 151.64/151.60 (E/Z), 150.22/150.10/149.99 (E/Z), 137.57/137.39 (E/Z), 137.00/136.72 (E/Z), 132.13/131.98 (E/Z), 131.29, 130.69/130.64 (E/Z), 128.62/128.46 (E/Z), 127.11/126.93 (E/Z), 121.01/120.94 (E/Z), 115.41/115.38 (E/Z), 112.97/112.84 (E/Z).

HRMS (ESI+) m/z: calculated for C₁₅H₁₀BrN₂O₄+ (M+H)⁺: 360.9746; found: 360.9818

### 1-(3-fluorophenyl)-5-(furan-2-ylmethylene)-2-thioxodihydropyrimidine-4,6(1H,5H)-dione 6m

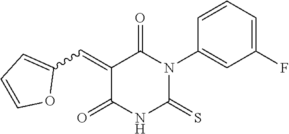 [SMILES: O=C1NC(=S)N(c2cccc(F)c2)C(=O)C1=Cc1ccco1]

30 mg (0.13 mmoles) of 2g and furan-2-carbaldehyde (12 μL, 1.1 eq) were dissolved in 1.5 mL of acetic acid. Reaction time=45 minutes. Yellow powder, Yield: 10 mg, 25% (E/Z mixture; 50:50).

¹H NMR (500 MHZ, THF-d₃) δ ppm: 11.99 (s, 1H)/11.93 (s, 1H) (E/Z), 8.79 (dt, J=3.8, 0.5 Hz, 1H)/8.60 (dt, J=3.9, 0.5 Hz, 1H) (E/Z), 8.36 (s, 1H)/8.29 (s, 1H) (E/Z), 8.12 (dd, J=1.7, 0.6 Hz, 1H)/8.11 (dd, J=1.7, 0.6 Hz, 1H) (E/Z), 7.55-7.41 (m, 2H), 7.24-7.14 (m, 2H), 7.13-7.03 (m, 4H), 6.87 (ddd, J=3.8, 1.7, 0.8 Hz, 1H)/6.81 (ddd, J=3.9, 1.7, 0.9 Hz, 1H) (E/Z).

¹³C NMR (126 MHZ, THF-d₈) δ ppm: 180.44/180.35 (E/Z), 163.90 (d, J_{C-F}=245.3 Hz)/163.86 (d, J_{C-F}=245.3 Hz) (E/Z), 162.58/160.97 (E/Z), 160.90/159.77 (E/Z), 152.43/152.28 (E/Z), 152.39/152.32 (E/Z), 141.83 (d, J_{C-F}=30.6 Hz)/141.74 (d, J_{C-F}=30.8 Hz) (E/Z), 140.05/139.70 (E/Z), 130.81 (d, J_{C-F}=8.4 Hz), 128.74, 126.46/126.29 (E/Z), 117.81 (d, J=19.6 Hz)/117.62 (d, J=19.6 Hz) (E/Z), 116.09, 115.92, 114.04 (d, J=22.7 Hz).

¹⁹F NMR (471 MHZ, THF-d₈) δ ppm: −114.19/−114.20 (E/Z).

HRMS (ESI+) m/z: calculated for C₁₅H₁₀FN₂O₃S⁺ (M+H)⁺: 317.0318; found: 317.0393.

### Example 2: Inhibitory Effect Against IFN-γ and/or Formin-Mediated Actin Assembly

### Materials and Methods

Cell culture: Hela cells were grown at 37° C. under 5% CO₂ in DMEM high glucose Glutamax (Gibco, Life Technologies) complemented with 10% FBS (v/v) (Gibco, Life Technologies) and supplemented with 5 mM pyruvate (v/v) (Gibco, Life Technologies) and 1% penicillin-streptomycin (v/v) (Gibco, Life Technologies). Primary antibodies: Mouse anti-phospho-STAT1 Tyr701 (BD Transduction Laboratories, 612132, RRID: AB_399503, 1:1000 for western blot), rabbit anti-STAT1 (Cell Signaling, 9172, RRID: AB_2198300, 1:1000 for western blot), mouse anti-alpha-tubulin (Sigma, clone B512, T5168, RRID: AB_86546, 1:5000 for western blot); rabbit anti-IFN-γ ([Abcam, EPR1108, ab133566, 1:1000 for western blot). Secondary antibodies anti-mouse-Alexa488 (Invitrogen, A21202), anti-mouse-HRP (Jackson ImmunoResearch, 715-035-151) and anti-rabbit-HRP (Jackson ImmunoResearch, 715-035-152) were used at 1:5000 for western blot.

### 1. JAK/STAT Signalling Activation Assay

20 mM stock solution of compounds in DMSO were prepared and stored at −20° C.

Hela cells were treated 20 min at 37° C. with mixture containing 1000 U·ml⁻¹ IFNγ in DMEM with 0.2% BSA preincubated for 20 min at 37° C. with DMSO (as control) for JAK/STAT stimulation. For screening compounds, 40 μM compounds were pre-incubated with 1000 U·ml⁻¹ of IFNγ in DMEM containing 0.2% BSA for 20 min at 37° C. before adding it to Hela cells. 10-fold 7 or 5 serial dilutions from 400 μM of compounds were made in DMEM containing 1000 U·ml⁻¹ of IFNγ and 0.2% BSA, and incubated for 20 min before stimulating JAK/STAT for finding IC50 towards IFNγ.

For comparing pSTAT1 status in different conditions of 5k treatment, JAK/STAT stimulation on Hela cells were made by adding mixture containing 1000 U·ml⁻¹ IFNγ in DMEM with 0.2% BSA and 40 UM compound pre-incubated for 20 min at 37° C.; similar mixture without pre-incubation; 40 μM compound in DMEM for 20 min at 37° C., then followed by addition of 1000 Ul·ml⁻¹ IFNγ to the media, and stimulated the JAK/STAT signaling for 20 min further.

### 2. Immunoblotting

Cells were lysed in sample buffer (62.5 mM Tris/HCl, pH 6.0, 2% v/v SDS, 10% glycerol v/v, 40 mM dithiothreitol, and 0.03% w/v phenol red). Samples were analysed by SDS-PAGE on 4-15% Mini-PROTEAN* TGX™ Precast Gels or on 4-15% Mini-PROTEAN′ TGX™ Stain Free Gel (Bio-Rad) and immuno-blotted with the indicated primary antibodies and horseradish peroxidase- or Alexa488-conjugated secondary antibodies. Chemiluminescence signal was revealed using Pierce ECL Western Blotting Substrate, SuperSignal West Dura Extended Duration Substrate or SuperSignal West Femto Substrate (Thermo Scientific Life Technologies). Acquisition and quantification were performed with the ChemiDoc MP Imaging System (Bio-Rad). Phosphorylated protein over total protein ratio was determined on the same blot using horseradish peroxidase and Alexa488 signals.

### 3. In Cellulo Actin Nucleation Assay

Hela cells grown on coverslips were treated with DMEM containing 0.2% BSA and the compounds (40 μM) for 20 min at 37° C., washed with cold PBS (two times) and then fixed with 4% paraformaldehyde for 30 min at room temperature, quenched in 50 mM NH₄Cl for 10 min and permeabilized with 0.05% saponin in 0.2% BSA in PBS for 20 min. Cells were incubated with 165 nM phalloidin for 1 h at room temperature. DAPI containing fluoromount-G was used to mount coverslips onto glass slide. Cell areas were measured with ImageJ software (NIH).

### 4. Cell Viability Assay

Cell viability assay was carried out by plating 10,000 cells/well in 96-well plates. 3-Fold 8 serial dilutions of the compounds from 3 mM were made in DMEM. HeLa cells were treated for 24 h with the compounds of different concentrations made. In case of preincubation with IFNγ, 6000 U·ml⁻¹ of IFNγ per concentration of compounds were used and incubated for 20 min. According to manufacturer's protocol,

CellTiter-Blue® reagent was added after 24 h treatment and cells were incubated for 3 h before recording fluorescence intensities (λex. 560/20 nm; λem. 590/10 nm) using a Perkin Elmer Wallac 1420 Victor2 Microplate Reader.

### Results

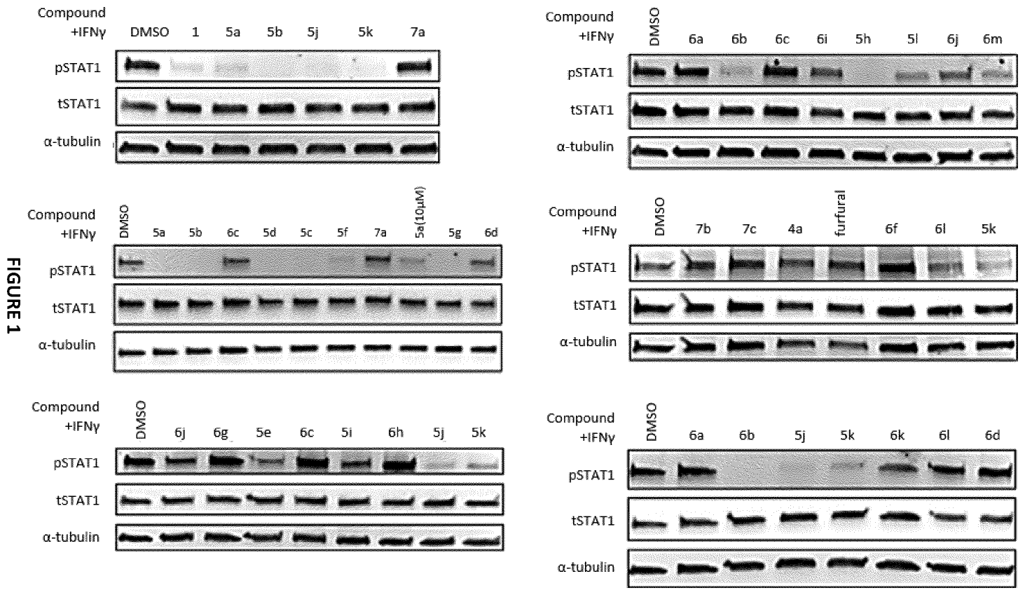

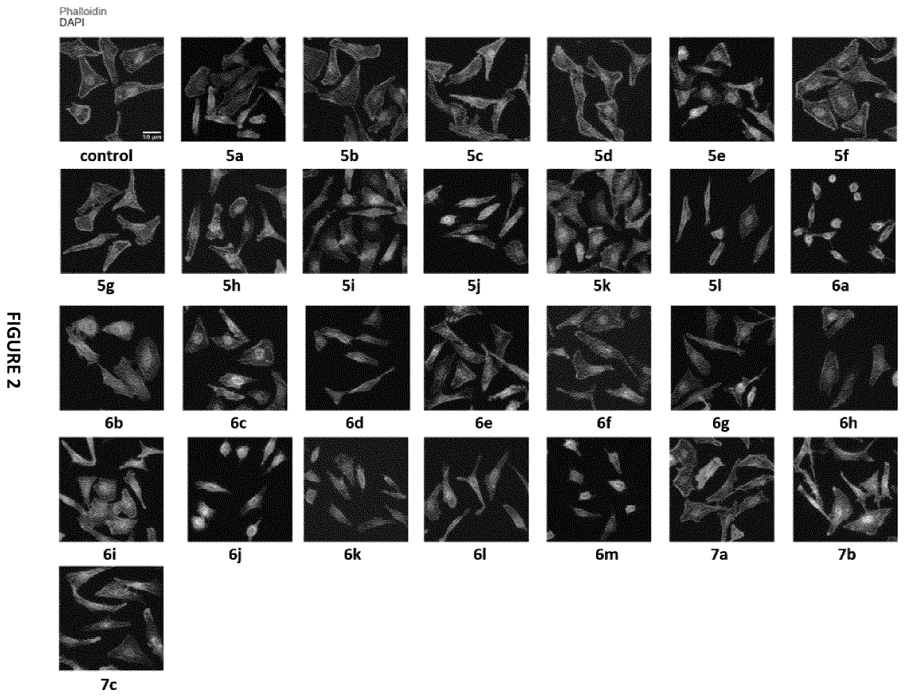

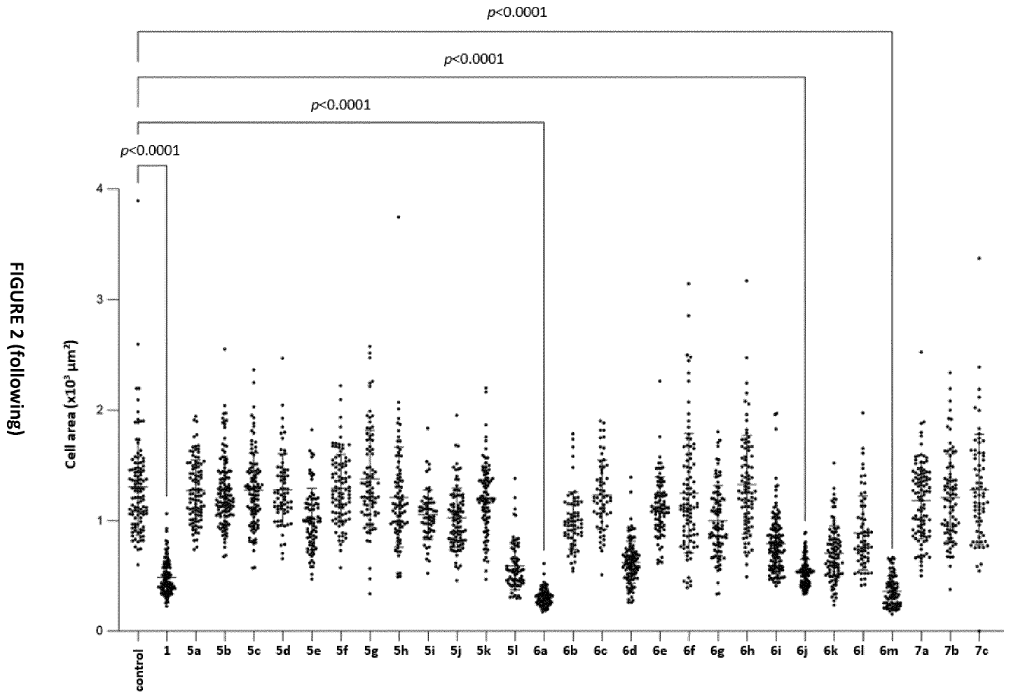

The analogs were independently evaluated for their capacity to inhibit IFNγ-induced tyrosine phosphorylation of STAT1 (pSTAT1) by western blotting and to alter actin assembly, monitoring phalloidin staining as well as measuring cell area, which is reduced in cells with impaired actin networks. Using HeLa cells as a suitable model to study IFNγ-signaling, the inventors found that most analogs that contain a terminal alkyne on the furane ring, including 5a, inhibited IFNγ-induced pSTAT1 but was deprived of formin targeting according to the phalloidin pattern and overall cell area that remained unaffected, characteristic of a normal actin network (FIGS. 1, 2). This indicated that inhibition of IFNγ-signaling occurs independently of formin targeting. Conversely, compounds containing a furane ring functionalized with chlorine, nitro or pyrrolidine substituents, analog 6h containing a furane ring attached to the thiobarbiturate core at a different position, compound 6d containing a thiophene instead of the furane, or 6l with a barbiturate core did not show IFNγ-signaling inhibitory activity. Additionally, analogs lacking α,β-unsaturated C—C bond such as 7a did not show any activity against IFNγ-signaling or actin assembly, suggesting that putative biological targets may react with the inhibitors by forming a covalent adduct. Similarly, compound 6g containing an additional unsaturation between furane and thiobarbiturate rings was deprived of biological activity in these assays. Furthermore, the inventors found that varying the nature and positions of the substituents on the aromatic ring was tolerated with no loss of activity. Interestingly, functionalizing thiobarbiturate with a second aromatic substituent identical to that already in place afforded biologically active compounds such as 5j and 5k, enabling us to investigate IFNγ-signaling using pure products as opposed to a mixture of stereoisomers. Thus, this synthetic strategy afforded a series of biologically active compounds, whose structure are inherently refractory to giving rise to distinct isomers upon rehydration/dehydration in biological settings. Surprisingly, the alkyne-containing barbiturate 5l retained its activity against both IFNγ-signaling and formin, whereas 6l did not inhibit the former pathway and all the other alkyne analogs were deprived of formin inhibitory properties. Furthermore, compounds 6a, 6b, and 6i containing furane rings functionalized at the same position but with distinct substituents inhibited either IFNγ-signaling or formins, showing that subtle modifications of the furane ring drastically impact on potential inhibitory effect. Nevertheless, this synthetic route allowed us to produce biologically active pure products such as 5k and 6j that can target either IFNγ-signaling or the formins, respectively, as well as negative controls 7a and 7b.

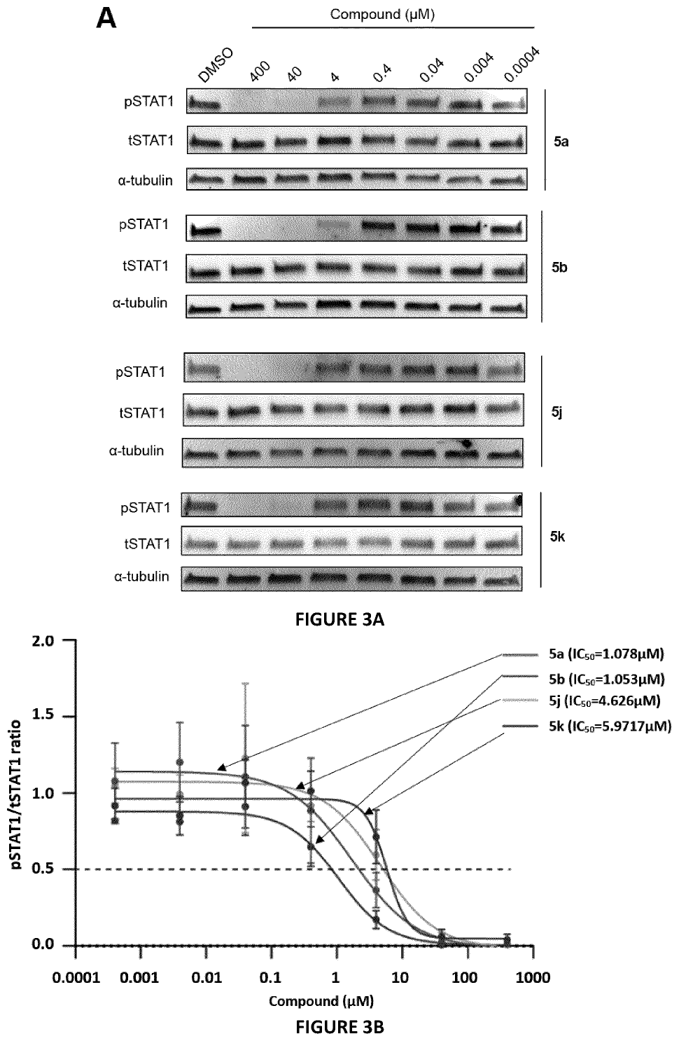

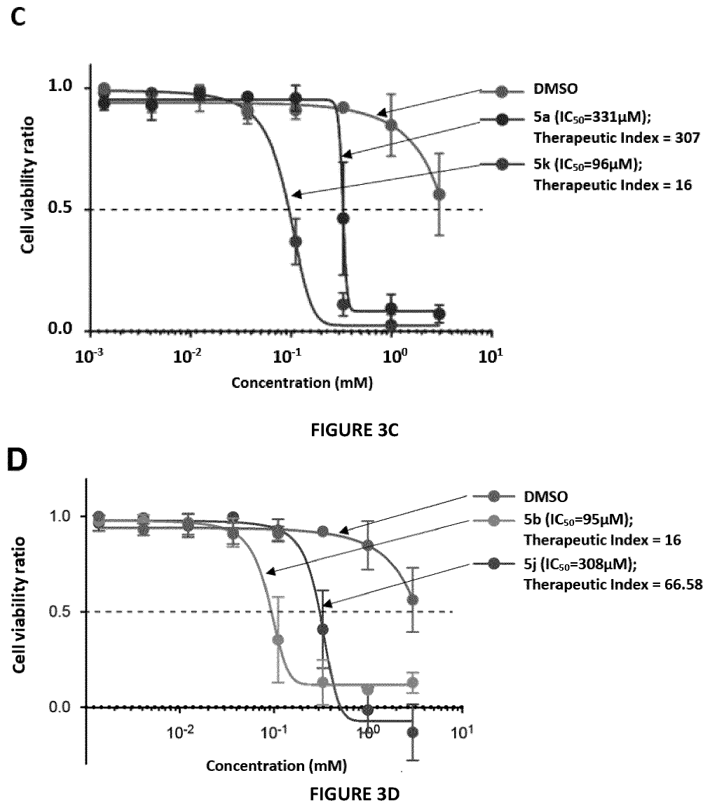

The inventors also explored the capacity of a subset of analogs to inhibit IFNγ-signaling selectively. They found that compounds 5a, 5b, 5j and 5k inhibited phosphorylation of STAT in a dose-dependent manner in the low micromolar range with 5a and 5b being slightly more potent, although used as a mixture of stereoisomers (FIG. 3A-B). Importantly, these compounds did not show significant cytotoxicity at effective doses (FIG. 3C-D).

TABLE 5

| | IC₅₀ pSTAT1 | IC₅₀ Cell | Therapeutic |
|---|---|---|---|
| Compound | inhibition (μM) | viability (μM) | index |
| 5a | 1.078 | 331 | 307 |
| 5b | 1.053 | 95 | 16 |
| 5j | 4.626 | 308 | 66.58 |
| 5k | 5.972 | 95 | 16 |

1-20. (canceled)

21. A compound of the following general formula (I): 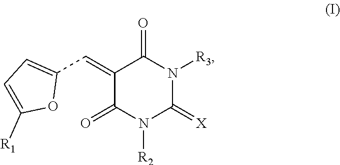
or a pharmaceutically acceptable salt, stereoisomer, tautomer or solvate thereof,
wherein,
X is an oxygen or sulfur atom;
R₁ is a chlorine atom or a (C₂-C₆)alkynyl group;
R₂ and R₃ are, independently of one another, a hydrogen atom, a (C₀-C₆)alkyl-ethynyl (—(CH₂)₀₋₆—C≡CH); a (C₀-C₃)alkyl-NH—C(O)—R′; a (C₀-C₃)alkyl-C(O)—NR′R″; a (C₀-C₃)alkyl-NH—C(O)—OR′; a (C₀-C₃)alkyl-NH—C(O)—NR′R″; a (C₀-C₃)alkyl-C(O)—R′; a (C₀-C₃)alkyl-C(O)—OR′; a (C₀-C₃)alkyl-NR′R″; a (C₀-C₃)alkyl-SOR′; a (C₀-C₃)alkyl-SO₂R′; a (C₀-C₃)alkyl-SONR′R″; a (C₀-C₃)alkyl-SO₂NR′R″; (C₀-C₃)alkyl-NHSO₂R′; or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)heterocycloalkyl group, (C₆-C₁₂)aryl group, or (C₅-C₁₂)heteroaryl group; said group being optionally substituted by at least one R;
R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group; and
R′ and R″ are independently selected from the group consisting of a hydrogen atom or a (C₁-C₆)alkyl group optionally substituted by at least one halogen;
provided that said compound is not a compound (Ia) or (Ib):

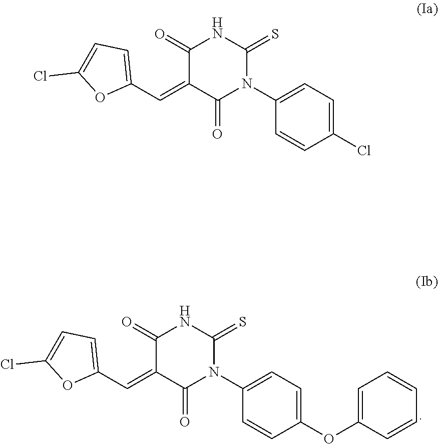 [SMILES: O=C1NC(=S)N(c2ccc(Cl)cc2)C(=O)/C1=C/c1ccc(Cl)o1 ; O=C1NC(=S)N(c2ccc(Oc3ccccc3)cc2)C(=O)/C1=C/c1ccc(Cl)o1]

22. The compound according to claim 21, wherein R₁ is a chlorine or an ethynyl group.

23. The compound according to claim 22, wherein R₁ is an ethynyl group.

24. The compound according to claim 21, wherein X is a sulfur atom.

25. The compound according to claim 21, wherein R₂ and R₃ are, independently of one another, a hydrogen atom or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)heterocycloalkyl group, (C₆-C₁₂)aryl group, and (C₅-C₁₂)heteroaryl group; said group being optionally substituted by at least one R where R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group provided that R₃ is not a hydrogen atom.

26. The compound according to claim 21, wherein R₂ is H or a (C₆-C₁₂)aryl group optionally substituted by at least one R; and R₃ is a (C₆-C₁₂)aryl group optionally substituted by at least one R.

27. The compound according to claim 21, wherein R₃ is a phenyl group substituted by at least one R.

28. The compound according to claim 21, wherein R₂ and R₃ are identical.

29. The compound according to claim 21, wherein the compound is selected from the group consisting of: 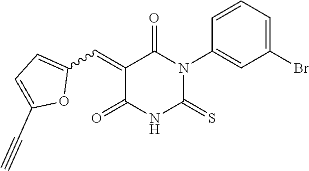 [SMILES: C#Cc1ccc(C=C2C(=O)NC(=S)N(c3cccc(Br)c3)C2=O)o1]
Compound 5a
(1-(3-bromophenyl)-5-((5-
ethynylfuran-2-yl)methylene)-2-
thioxodihydropyrimidine-4,6(1H,5H)-
dione) 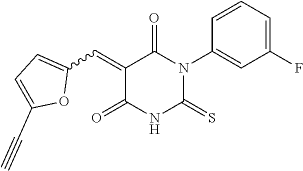 [SMILES: C#Cc1ccc(C=C2C(=O)NC(=S)N(c3cccc(F)c3)C2=O)o1]
Compound 5b
5-((5-ethynylfuran-2-
yl)methylene)-1-(3-
fluorophenyl)-2-
thioxodihydropyrimidine-
4,6(1H,5H)-dione 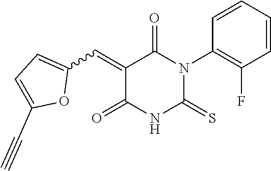 [SMILES: C#Cc1ccc(C=C2C(=O)NC(=S)N(c3ccccc3F)C2=O)o1]
Compound 5c
5-((5-ethynylfuran-2-
yl)methylene)-1-(2-
fluorophenyl)-2-
thioxodihydropyrimidine-
4,6(1H,5H)-dione 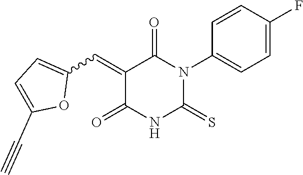 [SMILES: C#Cc1ccc(C=C2C(=O)NC(=S)N(c3ccc(F)cc3)C2=O)o1]
Compound 5d
5-((5-ethynylfuran-2-yl)methylene)-1-
(4-fluorophenyl)-2-
thioxodihydropyrimidine-4,6(1H,5H)-
dione 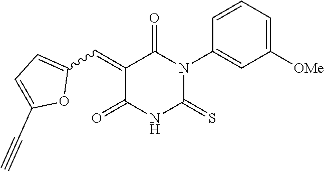 [SMILES: C#Cc1ccc(C=C2C(=O)NC(=S)N(c3cccc(OC)c3)C2=O)o1]
Compound 5f
5-((5-ethynylfuran-2-
yl)methylene)-1-(3-
methoxyphenyl)-2-
thioxodihydropyrimidine-
4,6(1H,5H)-dione 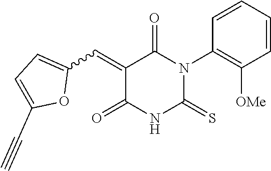 [SMILES: C#Cc1ccc(C=C2C(=O)NC(=S)N(c3ccccc3OC)C2=O)o1]
Compound 5g
5-((5-ethynylfuran-2-
yl)methylene)-1-(2-
methoxyphenyl)-2-
thioxodihydropyrimidine-
4,6(1H,5H)-dione 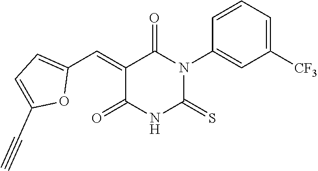 [SMILES: C#Cc1ccc(/C=C2\C(=O)NC(=S)N(c3cccc(C(F)(F)F)c3)C2=O)o1]
Compound 5h
5-((5-ethynylfuran-2-yl)methylene)-2-
thioxo-1-(3-
(trifluoromethyl)phenyl)dihydro-
pyrimidine-4,6(1H,5H)-dione 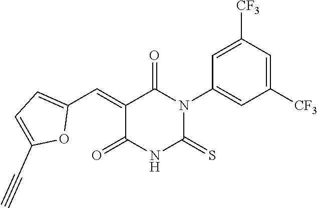 [SMILES: C#Cc1ccc(/C=C2\C(=O)NC(=S)N(c3cc(C(F)(F)F)cc(C(F)(F)F)c3)C2=O)o1]
Compound 5i
1-(3,5-
bis(trifluoromethyl)phenyl)-5-
((5-ethynylfuran-2-
yl)methylene)-2-
thioxodihydropyrimidine-
4,6(1H,5H)-dione 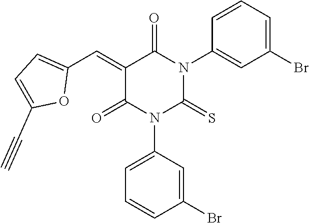 [SMILES: C#Cc1ccc(C=C2C(=O)N(c3cccc(Br)c3)C(=S)N(c3cccc(Br)c3)C2=O)o1]
Compound 5j
1,3-bis(3-bromophenyl)-5-
((5-ethynylfuran-2-
yl)methylene)-2-
thioxodihydropyrimidine-
4,6(1H,5H)-dione 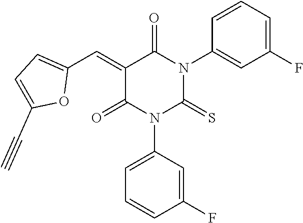 [SMILES: C#Cc1ccc(C=C2C(=O)N(c3cccc(F)c3)C(=S)N(c3cccc(F)c3)C2=O)o1]
Compound 5k
5-((5-ethynylfuran-2-yl)methylene)-
1,3-bis(3-fluorophenyl)-2-
thioxodihydropyrimidine-4,6(1H,5H)-
dione 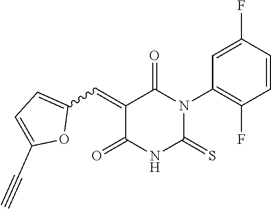 [SMILES: C#Cc1ccc(C=C2C(=O)NC(=S)N(c3cc(F)ccc3F)C2=O)o1]
Compound 5e
1-(2,5-difluorophenyl)-5-((5-
ethynylfuran-2-yl)methylene)-
2-thioxodihydropyrimdiine-
4,6(1H,5H)-dione 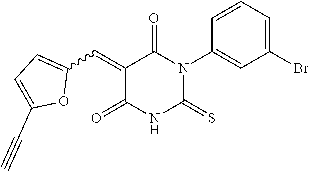 [SMILES: C#Cc1ccc(C=C2C(=O)NC(=S)N(c3cccc(Br)c3)C2=O)o1]
Compound 5l
1-(3-bromophenyl)-5-((5-
ethynylfuran-2-
yl)methylene)pyrimidine-
2,4,6(1H,3H,5H)-trione 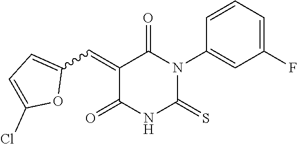 [SMILES: O=C1NC(=S)N(c2cccc(F)c2)C(=O)C1=Cc1ccc(Cl)o1]
Compound 6b
5-((5-chlorofuran-2-yl)methylene)-1-
(3-fluorophenyl)-2-
thioxodihydropyrimidine-4,6(1H,5H)-
dione 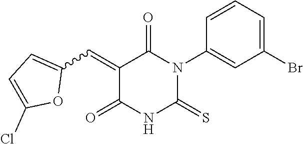 [SMILES: O=C1NC(=S)N(c2cccc(Br)c2)C(=O)C1=Cc1ccc(Cl)o1]
Compound 6c
5-((5-chlorofuran-2-
yl)methylene)-1-(3-
bromophenyl)-2-
thioxodihydropyrimidine-
4,6(1H,5H)-dione

30. A method of treating autoimmune and inflammation-associated diseases, viral diseases, atherosclerosis or metabolic syndrome, or cancer in a subject in need thereof comprising administering a compound according to claim 21 or a pharmaceutically acceptable salt, stereoisomer, tautomer or solvate thereof to the subject.

31. The method according to claim 30, said method treating a subject having a disease associated to the hyper-activation of interferon-γ mediated JAK/STAT signaling.

32. The method according to claim 30, said method treating a subject having haemophagocytic lymphohistiocytosis, Crohn's disease, systemic lupus erythematosus, psoriasis, rheumatoid arthritis, ulcerative colitis, or coronavirus diseases.

33. A method of inhibiting formin FH2 domains in a cell comprising contacting a cell with a compound selected from the group consisting of: 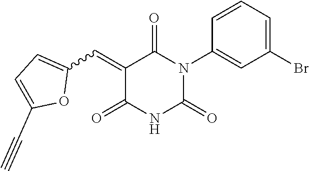 [SMILES: C#Cc1ccc(C=C2C(=O)NC(=O)N(c3cccc(Br)c3)C2=O)o1]
Compound 5l
1-(3-bromophenyl)-5-((5-
ethynylfuran-2-
yl)methylene)pyrimidine-
2,4,6(1H,3H,5H)-trione 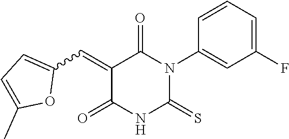 [SMILES: Cc1ccc(C=C2C(=O)NC(=S)N(c3cccc(F)c3)C2=O)o1]
Compound 6a
1-(3-fluorophenyl)-5-((5-
methylfuran-2-yl)methylene)-2-
thioxodihydropyrimidine-
4,6(1H,5H)-dione 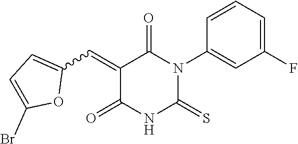 [SMILES: O=C1NC(=S)N(c2cccc(F)c2)C(=O)C1=Cc1ccc(Br)o1]
Compound 6i
5-((5-bromofuran-2-
yl)methylene)-1-(3-
fluorophenyl)-2-
thioxodihydropyrimidine-
4,6(1H,5H)-dione 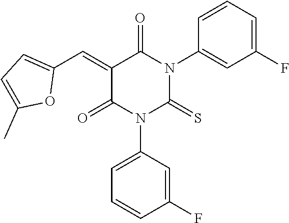 [SMILES: Cc1ccc(C=C2C(=O)N(c3cccc(F)c3)C(=S)N(c3cccc(F)c3)C2=O)o1]
Compound 6j
1,3-bis(3-fluorophenyl)-5-((5-
methylfuran-2-yl)methylene)-2-
thioxodihydropyrimidine-
4,6(1H,5H)-dione 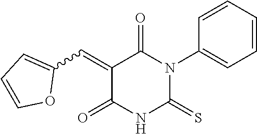 [SMILES: O=C1NC(=S)N(c2ccccc2)C(=O)C1=Cc1ccco1]
Compound 6k
5-(furan-2-ylmethylene)-1-
phenyl-2-
thioxodihydropyrimidine-
4,6(1H,5H)-dione 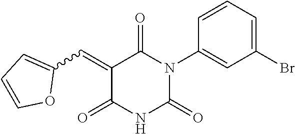 [SMILES: O=C1NC(=O)N(c2cccc(Br)c2)C(=O)C1=Cc1ccco1]
Compound 6l
1-(3-bromophenyl)-5-(furan-2-
ylmethylene)pyrimidine-
2,,6(1H,3H,5H)-trione 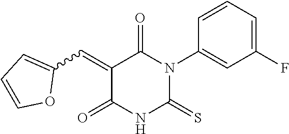 [SMILES: O=C1NC(=S)N(c2cccc(F)c2)C(=O)C1=Cc1ccco1]
Compound 6m
1-(3-fluorophenyl)-5-(furan-2-
ylmethylene)-2-
thioxodihydropyrimidine-
4,6(1H,5H)-dione

34. A method for inhibiting interferon-γ mediated signaling in a subject in need thereof, comprising administering a therapeutically effective amount of a compound of general formula (I) or a pharmaceutical composition comprising said compound to said subject, wherein said compound has general formula (I): 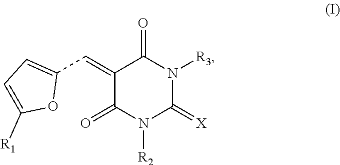
or is a pharmaceutically acceptable salt, stereoisomer, tautomer or solvate thereof,
wherein,
X is an oxygen or sulfur atom;
R₁ is a hydrogen atom, halo, nitro (NO₂), (C₁-C₆)alkyl group or (C₂-C₆)alkynyl group;
R₂ and R₃ are, independently of one another, a hydrogen atom, a (C₀-C₆)alkyl-ethynyl (—(CH₂)₀₋₆—C≡CH); a (C₀-C₃)alkyl-NH—C(O)—R′; a (C₀-C₃)alkyl-C(O)—NR′R″; a (C₀-C₃)alkyl-NH—C(O)—OR′; a (C₀-C₃)alkyl-NH—C(O)—NR′R″; a (C₀-C₃)alkyl-C(O)—R′; a (C₀-C₃)alkyl-C(O)—OR′; a (C₀-C₃)alkyl-NR′R″; a (C₀-C₃)alkyl-SOR′; a (C₀-C₃)alkyl-SO₂R′; a (C₀-C₃)alkyl-SONR′R″; a (C₀-C₃)alkyl-SO₂NR′R″; (C₀-C₃)alkyl-NHSO₂R′; or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)heterocycloalkyl group, (C₆-C₁₂)aryl group, or (C₅-C₁₂)heteroaryl group; said group being optionally substituted by at least one R;
R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group; and
R′ and R″ are independently selected from the group consisting of a hydrogen atom or a (C₁-C₆)alkyl group optionally substituted by at least one halogen.

35. The method according to claim 34, said method treating a subject having autoimmune and inflammation-associated diseases, viral diseases, atherosclerosis or metabolic syndrome, cancer, haemophagocytic lymphohistiocytosis, Crohn's disease, systemic lupus erythematosus, psoriasis, rheumatoid arthritis, ulcerative colitis, or coronavirus diseases.

36. The method according to claim 34, said method treating a subject having a disease associated to the hyper-activation of interferon-γ mediated JAK/STAT signaling.

37. The method according to claim 34, wherein R₁ is a halo selected from the group consisting of chlorine, fluorine and bromine, a methyl group or a (C₀-C₄)alkyl-ethynyl (—(CH₂)₀₋₄—C≡CH.

38. The method according to claim 34, wherein R₁ is a chlorine or an ethynyl group.

39. The method according to claim 34, wherein R₁ is an ethynyl group.

40. The method according to claim 34, wherein X is a sulfur atom.

41. The method according to claim 34, wherein R₂ and R₃ are, independently of one another, a hydrogen atom or a group selected from (C₁-C₆)alkyl group, (C₁-C₆)alkyloxy group, (C₂-C₆)alkenyl, (C₂-C₆)alkynyl, (C₃-C₁₀)cycloalkyl group, (C₃-C₁₀)heterocycloalkyl group, (C₆-C₁₂)aryl group, and (C₅-C₁₂)heteroaryl group; said group being optionally substituted by at least one R where R is independently selected from the group consisting of a halo, a hydroxyl, a thiol, a cyano, a nitro, an amino (—NH₂), a phosphate (PO₄³⁻), —CF₃, a (C₁-C₆)alkyl group, a (C₂-C₆)alkenyl, a (C₂-C₆)alkynyl, or a (C₁-C₆)alkyloxy group; provided that R₃ is not a hydrogen atom.

42. The method according to claim 34, wherein R₂ is H or a (C₆-C₁₂)aryl group optionally substituted by at least one R; and R₃ is a (C₆-C₁₂)aryl group optionally substituted by at least one R.
